(19) United States
(12) Patent Application Publication
(10) Pub. No.: US 2019/0221955 A1
(43) Pub. Date: Jul. 18, 2019

(54) BIOMETRIC BELT CONNECTOR

(71) Applicant: NOX MEDICAL, Reykjavik (IS)

(72) Inventors: Sveinbjörn HÖSKULDSSON, Reykjavik (IS); Kormakur HERMANNSSON, Reykjavik (IS)

(21) Appl. No.: 16/324,787

(22) PCT Filed: May 26, 2017
(86) PCT No.: PCT/IB2017/053128

§ 371 Date: Feb. 11, 2019

Related U.S. Application Data
(60) Provisional application No. 62/341,860, filed on May 26, 2016.

(51) Int. Cl. H01R 13/20; A61B 5/08; A61B 5/00; H01R 11/22; H01R 12/77; H01R 4/48

(52) U.S. Cl. CPC H01R 13/20; A61B 5/0806; A61B 5/6831; H01R 11/22; H01R 2201/12; H01R 4/48; A61B 2562/227; H01R 12/775; H01R 12/777

(57) ABSTRACT

An electrode belt connector is provided for electrically connecting a conductor of an electrode belt to a first snap portion of a snap connector electrode configured to be connected to a biometric device. The electrode belt connector includes a frame and an electrically conductive portion. The frame defines a corresponding second snap portion configured to mate with the first snap portion of the snap connector electrode. The electrode belt connector further includes a connecting portion configured to electrically connect the conductor of the electrode belt to the first snap portion of the snap connector electrode.

### FIELD OF THE DISCLOSURE

The present disclosure is within the field of medical devices, in particular biometric devices for measuring biosignals, and relates particularly to electrodes for such devices and in particular electrode belts and connectors for such belts.

### BACKGROUND

Electrode belts are known, both for direct contact galvanic electrodes used to measure cardiography signals and inductive belts used in respiratory inductive plethysmography. Such belts have various types of connectors, for transmitting the received signal to the respective device. There remains a need for improved belt connectors that are reliable and easy to use and maintain. Further, due to the signal measured by the electrode belt, there is a need for a reliable and very low-resistance electrical connection between conductor within the belt and an electrical connector of a mating biometric device.

### SUMMARY

The disclosure provides an electrode belt connector for electrically connecting a conductor of an electrode belt to a male snap portion of a snap connector electrode configured to be connected to a biometric device. The electrode belt connector includes a frame defining a receiving hole, the receiving hole being configured to receive a protrusion of the male snap portion of the snap connector electrode. The electrode belt connector further includes an electrically connective portion configured to electrically connect a wire of the electrode belt to the male snap portion of the snap connector.

### BRIEF DESCRIPTION OF THE FIGURES

FIGS. 1A, 1B, 1C, 1D, 1E, 1F, 1G, and 1H illustrate a male snap portion of snap connector according to an embodiment.

FIGS. 2A, 2B, 2C, and 2D illustrate a frame portion of snap connector according to an embodiment.

FIGS. 3A, 3B, 3C, 3D, 3E, 3F, 3G, 3H, 3I and 3J illustrate an electrode belt connector according to another embodiment.

FIGS. 4A, 4B, 4C, 4D, 4E, 4F, 4G, 4H, and 4I illustrate an electrode belt connector according to another embodiment.

FIGS. 5A, 5B, 5C, 5D, and 5E illustrate an electrode belt connector according to another embodiment.

FIG. 6 illustrates a conductive connector according to an embodiment.

FIGS. 7A, 7B, 7C, 7D, 7E, 7F, and 7G illustrate an electrode belt connector according to another embodiment.

FIGS. 8A and 8B illustrate conductive connectors according to other embodiments.

FIGS. 9A, 9B, 9C, 9D, 9E, 9F, 9G, 9H, and 9I illustrate an electrode belt connector according to another embodiment.

FIGS. 10A, 10B, 10C, 10D, and 10E illustrate an electrode belt connector according to another embodiment.

FIGS. 11A, 11B, 11C, 11D, 11E, and 11F illustrate an electrode belt connector according to another embodiment.

FIGS. 12A, 12B, 12C, 12D, 12E, 12F, and 12G illustrate an electrode belt connector according to another embodiment.

FIGS. 13A, 13B, 13C, and 13D illustrate an electrode belt connector according to another embodiment.

### DETAILED DESCRIPTION

The disclosure provides a belt connector for electrically connecting an electrode belt to a biometric device to be carried on a human or animal body. The belt connector may be made from one single piece which can be economically manufactured in order to function as a single-use consumable, to be used with a matching biometric device. The belt connector may comprise a plastic or a molded plastic frame having a front side and a rear side. The frame may have a receiving hole which has radial flexibility to function as a female snap button fastener to receive and fasten on the front side of the frame a male snap protrusion. The belt connector may further comprises fastening means for fastening to the frame a belt end of said electrode belt, and a member adjacent to said snap fastener receiving hole to engage an electrode wire end electrically connected to said belt such that said wire end is in electrical contact with said hole, either by extending into the hole or coming in electrical contact e.g. through a bridging conductor or a conductive connector, with a conducting male snap fastener inserted in said receiving hole.

The belt connector and belt may be configured such that a person wearing the belt under operation is insulated from current running through the belt, to meet existing standards for medical devices. The belt connector of the present disclosure is configured accordingly, and in an embodiment, the belt connector may comprises a shield member which is arranged on the rear side of said frame (toward the patient or carrier) to electrically shield the wire end from the rear side exterior of the belt connector.

The belt connector may comprise a cover enclosing the connector and wire end. The cover may suitably include a pre-perforated hole overlapping the hole of the frame, or in other embodiments is made from such material that can readily be perforated by pressing the connector onto a male fastener which fits the receiving hole of the frame.

The belt end may be fixedly engaged with the connector and the electrode wire connected to the connector such that the electrode wire is in electrical contact with the female snap fastener hole and thereby comes in electrical contact with a conducting male snap fastener inserted in the hole.

The belt end may be engaged with the belt connector in a fashion allowing adjustment of the length of the belt.

The disclosed belt connector is suitable for various types of electrode belts, such as for cardiographic measurements, both in clinical settings or for training purposes, but also for belts such as RIP (respiratory inductive plethysmography) belts.

As mentioned above, the belt connector is intended for electrically connecting an electrode belt to a biometric device, the term biometric device in this context includes any device that receives electrical biosignals as well as extension cables, intermediate devices, connecting boxes, etc. or other means for receiving and transmitting the biosignals.

The belt connector may be made from any of various suitable non-conducting materials, including, but not limited to, polymers or plastic materials, such as but not limited to ABS (acrylonitrile butadiene styrene), PC/ABS, polyethylene, e.g. low density polyethylene (LDPE) or high density polyethylene (HDPE), or derivatives such as polyethylene terephthalate (PET) or polyfluoroethylene (PTFE), or polypropylene, polyvinyl chloride, or polyamide (nylon). In other embodiments the connector may be made from paper based material or other material from natural fibers or a rubber material.

The electrode belt is generally a flexible belt such as commonly used in respiratory inductance plethysmography (RIP) devices. Such an electrode belt may be a flexible textile belt where an electrode wire is interwoven in the belt or can be laminated between two layers, typically in a zig-zag fashion to allow longitudinal elasticity.

The frame of the connector has a front side and a rear side, which are defined as follows: the front side of the frame faces the biometric device which is fastened onto the connector for operation and the rear side faces away from the device. In an embodiment, the rear side of the connector may face the body of the patient when mounted, i.e. the belt connector comes between the patient and the biometric device. However, the biometric device can also be configured such that the biometric device faces the patient and the belt connectors lie on top of the device, i.e. connects to the device on the face of the device facing away from the patient, thus in such embodiment the front side of the connectors face the patient and the rear side face away from the patient.

The frame may have or define a receiving hole with radial flexibility to function as a female snap button fastener for receiving and fastening on the front side of the frame a male snap protrusion. A mating biometric device may thus have a corresponding mating male snap fastener which can be fastened securely onto the belt connector. The hole can be shaped circular or semi-circular. Further, the hole may have a shape different from a circular or a semi-circular shape. For example, the hole may any other suitable shape, such as a general elongated shape shaped by two parallel members, suitably including guiding members to ensure proper positioning of the mating male snap member, a square opening, a rectangular opening, an elliptical opening, triangular opening, or a combination of any of such openings.

The receiving hole may have radial flexibility, which may be achieved by one or more slot extending from said hole.

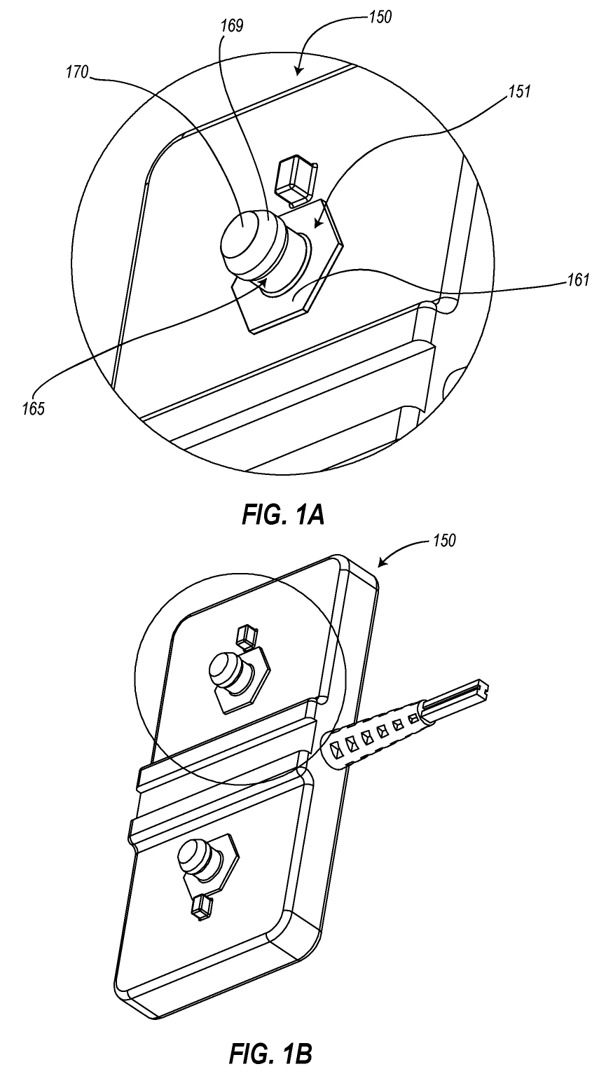

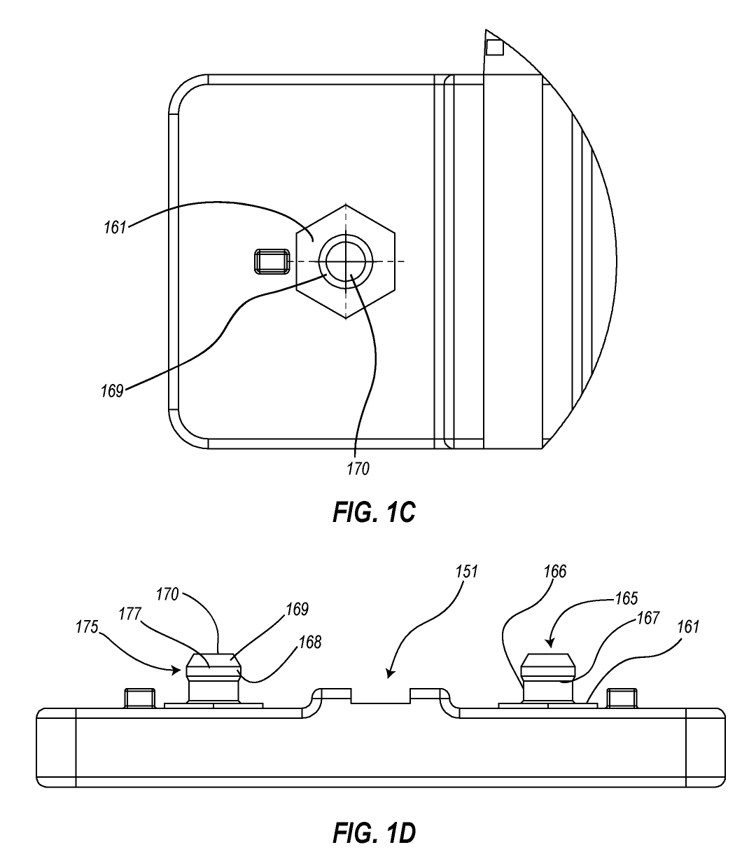

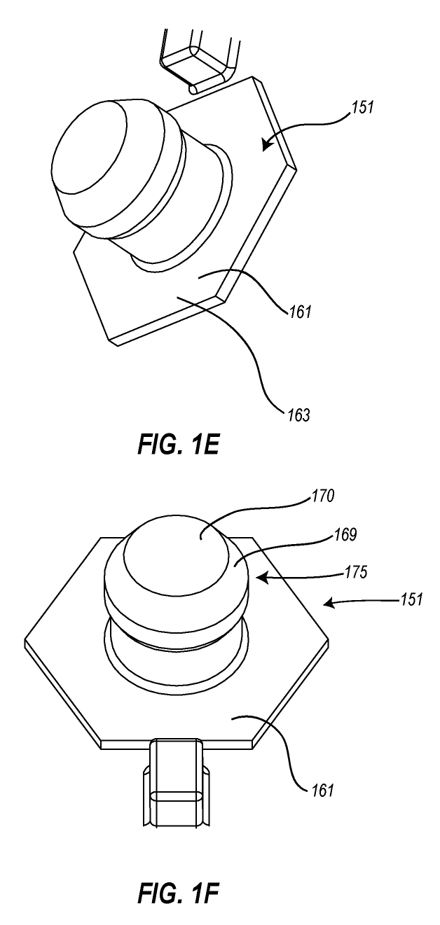

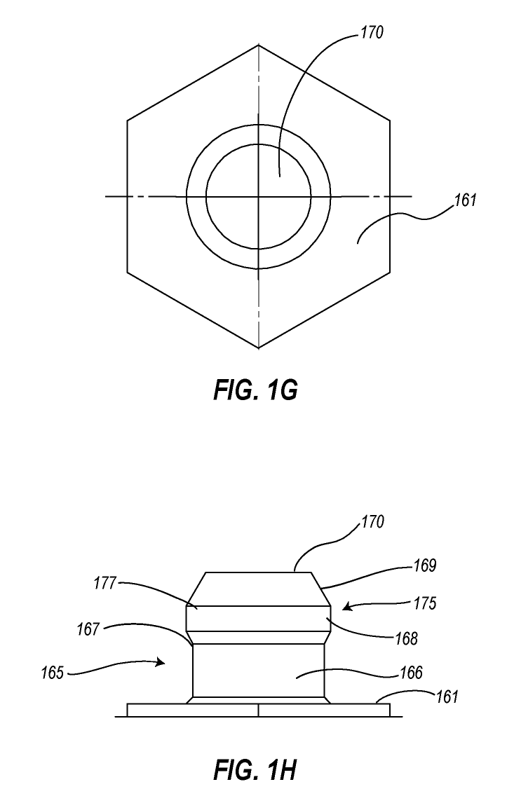

FIG. 1A shows an enlarged view of a portion of a biometric device 15 of FIG. 1B. As shown in FIG. 1A, a biometric device 150 may have a mating portion including male snap portion 151 of a snap connector. The male snap portion 151 is electrically connected to the internal electrical leads of the biometric device 150. As shown in FIGS. 1A to 1D, and in the enlarged drawings of FIGS. 1E to 1H, the male snap portion 151 may include a male snap foundation 161 from which a male snap protrusion 165 extends. As shown particularly in FIGS. 1D and 1H, the male snap protrusion may include a cylindrically shaped protrusion shaft 166 that extends perpendicular or nearly perpendicularly from the surface of male snap foundation 161. Protrusion shaft 166 may have a lateral surface 167. On a distal end of the protrusion shaft 166, the cross-sectional diameter of the male snap protrusion 165 may enlarge at the underside bevel 168, forming a bulged portion 175, the bulged portion 175 being formed at the distal end of the male snap protrusion 165. The underside bevel 168 may extend to a lateral surface 177 of the bulged portion, the lateral surface 177 having a cylindrical shape with a cross-sectional diameter greater than the protrusion shaft 166. The bulged portion may further have a top bevel 169 where the cross-sectional diameter reduces. A top surface 170 is formed adjacent to the upper edge of the top bevel 169. Male snap portion 151 is made of an electrically conductive material, including, but not limited to brass, copper, aluminium, steel, gold, or other metals or metal alloys, or even non-metal electrically conducting materials.

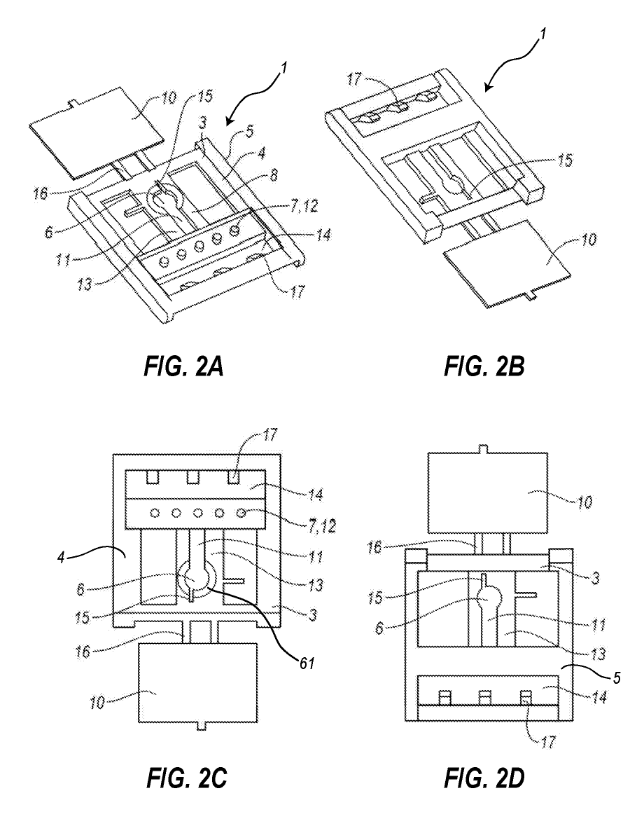

As shown in the embodiments of FIGS. 2A to 2D, a biometric belt connector 1 includes a frame 3 configured to be electrically connected to an electrode belt 2. The frame 3 may have a front side 4 and a rear side 5, a circular or semi-circular shaped receiving hole 6 with radial flexibility to function as a female snap button fastener. The receiving hole 6 may include a bevelled edge portion 61 on front side 4 to more easily receive and guide a male snap fastener into hole 6. The frame 3 may be a molded frame, and further may be made from a molded plastic material. Frame 3 may include fasteners 7, such as pins, which may comprise a ridge member 12. According to the first embodiment, the ridge member 12 may include a series of butts or pins 7 which are provided transverse or substantially to the belt direction and to which the belt end can be fastened onto with heat melting or gluing. The frame 3 may include two members, first member 8 and second member 13, that extend adjacent to hole 6. First member 8 and second member 13 may extend parallel to each other. Or in another embodiment, first member 8 may be arranged at an angle to second member 13. The two members 8, 13 may form a first slot 11 that extends from the hole 6. First member 8 and second member 13 may also form a second slot 15. Second slot may be formed across the hole 6 from the first slot 11.

Thus two slots 11 and 15 may extend across from each other in a direction substantially parallel to a direction of the belt to which the connector 1 is to be connected. The one or more slots may be formed or defined by at least one of the elongated members 8, 13. Members 8, 13 may have flexibility transverse to their longitudinal axis (e.g. by being sufficiently thin), thus imparting flexibility to the width of the hole 6. The hole 6 may thus be between two elongated members 8, 13 where one or both have sufficient and suitable flexibility to provide a snap fastener hole with suitable fastening strength.

Either one of first member 8 or second member 13, formed adjacent to said snap fastener receiving hole, may engage an electrode wire coming from the belt end. This wire may thus be pushed into electrical contact with a conducting male snap fastener provide in said receiving hole, either by extending into the hole, through the hole a single time or extending through the hole multiple times, for example, by being wrapped around one or both of the first member 8 or the second member 13, or coming in electrical contact with the hole, e.g., through a bridging conductor or conductive connector.

The elongated members 8, 13 and slots 11, 15 may be formed to provide the hole 6 with sufficient flexibility (i.e. elasticity in the width of the hole) to function as a female snap fastener. The member 13 may also function to engage an electrode wire end 9 from the belt end electrically connecting the belt 2 with the hole 6 and which comes in electrical contact with a conducting male snap fastener inserted in said hole. The connector further comprises a belt slot 14 with teeth members or pins 17, through which slot a loop of said belt 2 can be inserted such that it is held by the teeth/pins when pulled back, to adjust the length of the belt.

The connector further comprises a shield member 10 which may be molded in one piece with the frame 3 and joined to the frame with foldable hinges 16 such that the shield member 10 can be folded over to cover the rear side 5 of the hole 6 and wire end 9.

In one embodiment, the wire end may be crimped onto said member such that the crimping tubing fixes the wire and conducts and connects electrically the wire to the receiving hole, such that thus the wire and the belt is in electrical contact with a conducting male snap fastener inserted in said receiving hole.

The slot mentioned above can also function to provide an additional opening for a mating male projection on the biometric device. By this arrangement it is assured that the device cannot be (or is unlikely to be) incorrectly fastened, and the device will not fit any generic non-proprietary belts having connectors with female fasteners but without the correctly shaped and placed extended hole.

The connector frame has in another embodiment a separate further hole, not joined to the main fastener and electrical connection hole, where the further hole can mate with a corresponding male projection on said biometric device. Alternatively, the biometric device can have a female hole for mating with a corresponding male projection on the belt connector.

The connector frame further comprises a fastener to fasten the frame to a belt end of an electrode belt. The fastener can in one embodiment comprise a slot with a row of teeth, pins or hooks, transverse to the belt direction, to engage a belt end. The belt slot provides for insertion of a loop of the belt through the belt slot to be held securely by the row of teeth, pins, or hooks, such that the belt length is adjusted and fixed, so that a user can later re-adjust the length.

In another embodiment, the fastening means comprise a ridge member, which can be a flat or sharp elongated ridge or ridge or row comprising pins or hooks, which ridge lies transverse to the belt direction and to which a belt end can be fastened onto with heat melting or gluing. Alternatively, the ridge member can have pins hooks that grab onto the belt fabric without need of heating.

The frame may also have an adjustment slot for user adjustment of the belt, which can configured with either of the two described fastening means, the adjustment slot having a row of teeth, pins or hooks transverse to the belt direction, through which adjustment slot a loop of said belt can be inserted, which hooks onto the teeth/pins when pulled on, such that the length of the belt can be readily adjusted but also secured in the desired adjusted length.

The connector may include a cover substantially or essentially fully enclosing the frame, which cover either includes a pre-made hole overlapping the receiving hole of the frame, or can be readily perforated by pressing the connector onto a male fastener which fits the receiving hole of the frame. A suitable cover can be arranged by a suitably sized paper, plastic or fabric sticker (foldable sheet with glue on one side) which sticker is folded over the frame after the belt end has been fastened and the wire end electrically connected to the receiving hole, or the cover can be from but not limited to a paper envelope, a plastic envelope and a textile envelope, which envelope is suitably fastened by gluing, sewing or the like.

In the embodiments where the biometric device has a further male projecting member which fits within the slot of the frame or within a separate mating hole, the cover is suitably arranged with corresponding openings for such hole or slot for receiving such mating male member, and the cover may also have a suitable hole allowing the protrusion of a male protruding member being a part of the frame which fits in a mating receiving hole or slot on the mating biometric device.

In an embodiment, the connector comprises a shield member which is arranged on the rear side of the frame to electrically shield the wire from the rear side exterior of the belt connector. The shield member is in one embodiment a sheet member extending from the frame, which sheet member is configured to be folded over onto the rear side of the frame to cover said hole and engaged wire. Such shield member molded in one piece with the frame with enough strength but suitably flexible to allow folding at least once without braking allows the use of a cover enclosing the frame, which cover need not be electrically insulating, as the shield insulates the only part of the connector which could conduct electrical current of the connector, except through the hole.

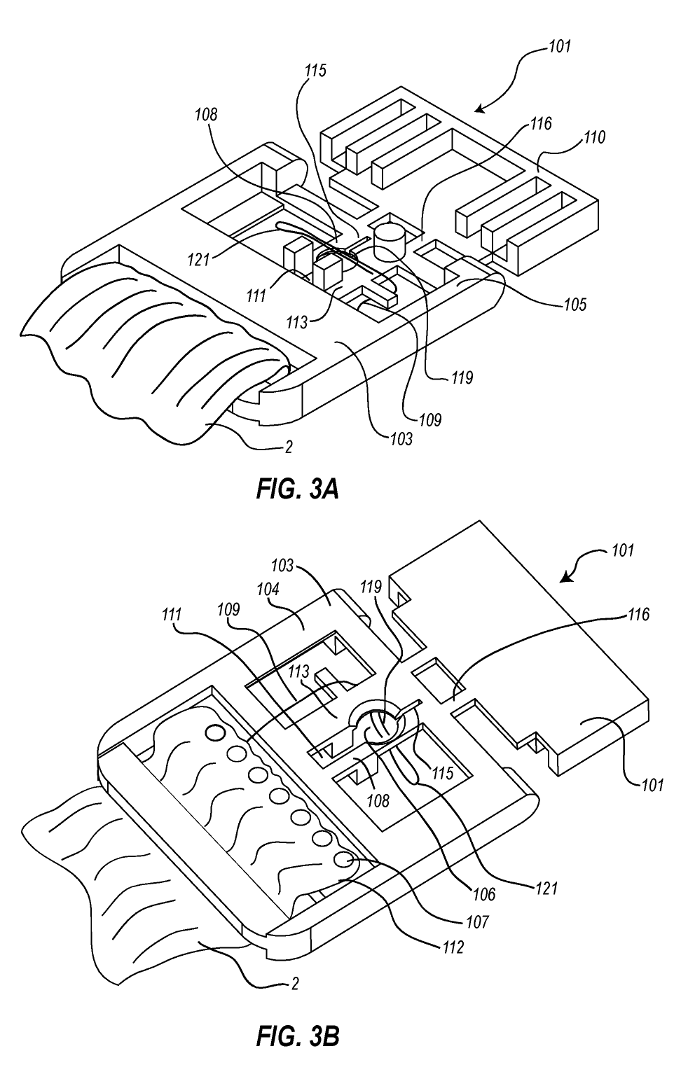

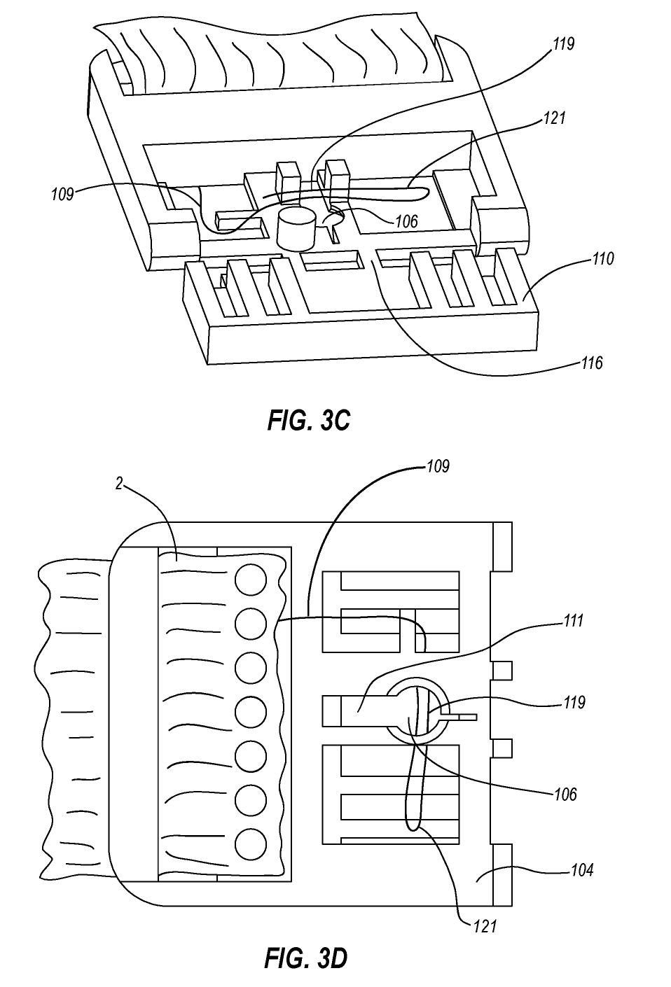

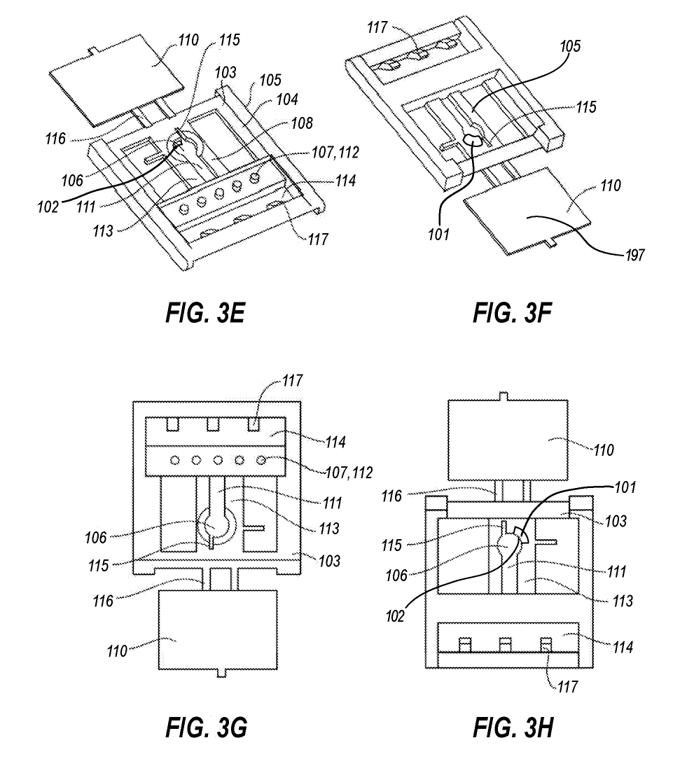

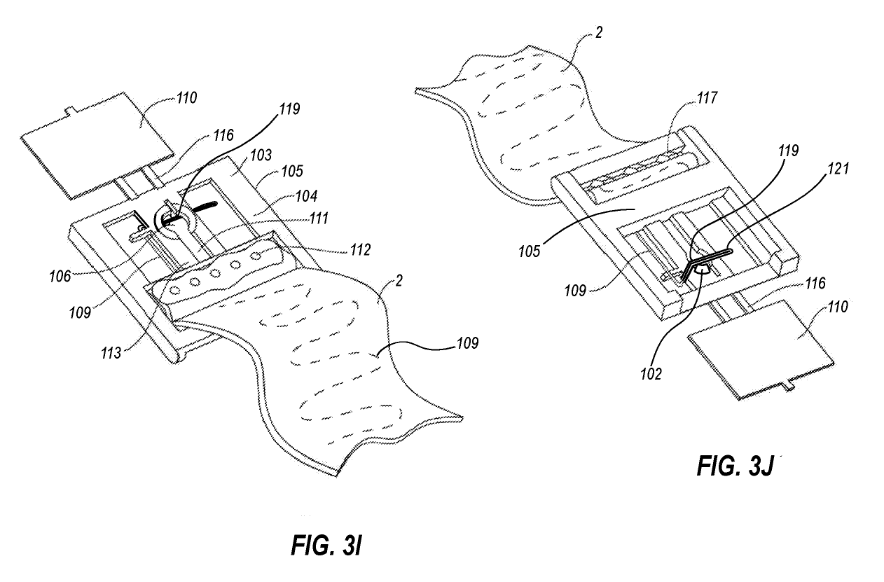

According to another embodiment illustrated in FIGS. 3A to 3J, instead of threading an end portion of the electrode wire through the receiving hole, an end portion 109 of an electrode wire from belt 2 is arranged to extend across or bypass a portion of the receiving hole 106 defined in frame 103 by first member 108 and second member 113. Thus, the end portion of electrode wire 109 is arranged to extend across the receiving hole 106 on the rear side 105 of the frame 103 (the rear side 105 being the side of the frame 103 that is opposite of the frame from the front side 104 of the frame 103 that is positioned in contact with the biometric device).

According to the embodiments of FIGS. 3A to 3J, the belt connector 101 is configured such that when the male snap portion penetrates the hole 106, radial flexibility of first and second members 108 and 113 fasten the frame to the male snap portion, and upon fastening the frame to the male snap portion, a contact portion 119 of the wire 109 provided in belt 2 is forced into contact with a distal portion of the male snap portion, for example, the contact portion 119 of the wire is forced against the top surface 170 or a surface of the top bevel 169 of the male snap portion 151 or both. According to the embodiment of FIGS. 3A to 3J, a reliable electrical connection between the contact portion 119 and the male snap portion 151 can be secured. According to the embodiment, the electrode wire 109 can be formed into a looped portion 121 to provide additional (e.g. doubled) surface area as the contact portion 119 of the wire. Additionally, an inside surface of the shield 110 or an additional lid may provide structural support to force the contact portion 119 of the electrode wire into secure contact with the male snap portion 151.

The contact portion 119 of the electrode wire can be held in place by passing or extending across the receiving hole 106 through various means. For example, as shown in FIGS. 3E, 3F, and 3H, a contact member 101 may be formed adjacent or near hole 106, and contact member 101 may have a lateral surface 102 configured to exert a lateral force onto an area of contact portion 119 of the electrode wire. Thus, the contact portion 119 of the electrode wire can be maintained in position. Although not shown, multiple contact members 101 may be formed or defined by frame 103 to hold contact portion 119 of electrode wire 109 to extend across a rear side 105 of said frame 103. Similarly, although not shown, contact portion 119 may be held in place by an adhesive applied to a portion of first member 108 or second member 113. Or in another embodiment, a groove or a recess portion, or a plurality of grooves or recess can be formed in a portion of the frame adjacent to hole 106, for example, on the rear side 105 of the frame 103 at first member 108 or 113, or both. The groove(s) or recess portion(s) can be configured to at least partially envelop a portion of electrode wire 109 to maintain a contact portion 119 of the electrode wire to extend across or bypass the receiving hole 106. Alternatively, although not shown, a contact member, adhesive, or groove me similar be formed or applied to an inside surface 197 of shield 110 or an additional lid structure, to maintain the contact portion 119 in place against, for example, the top surface 170 of the male snap protrusion 151.

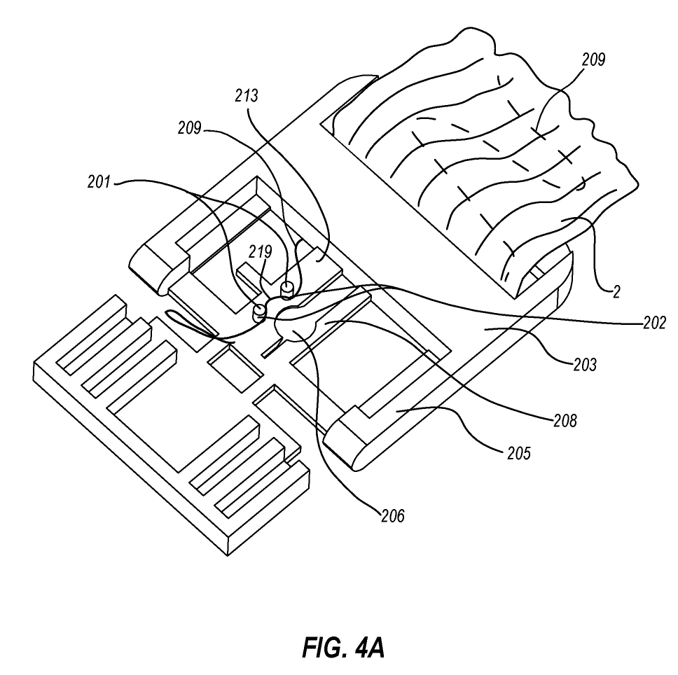

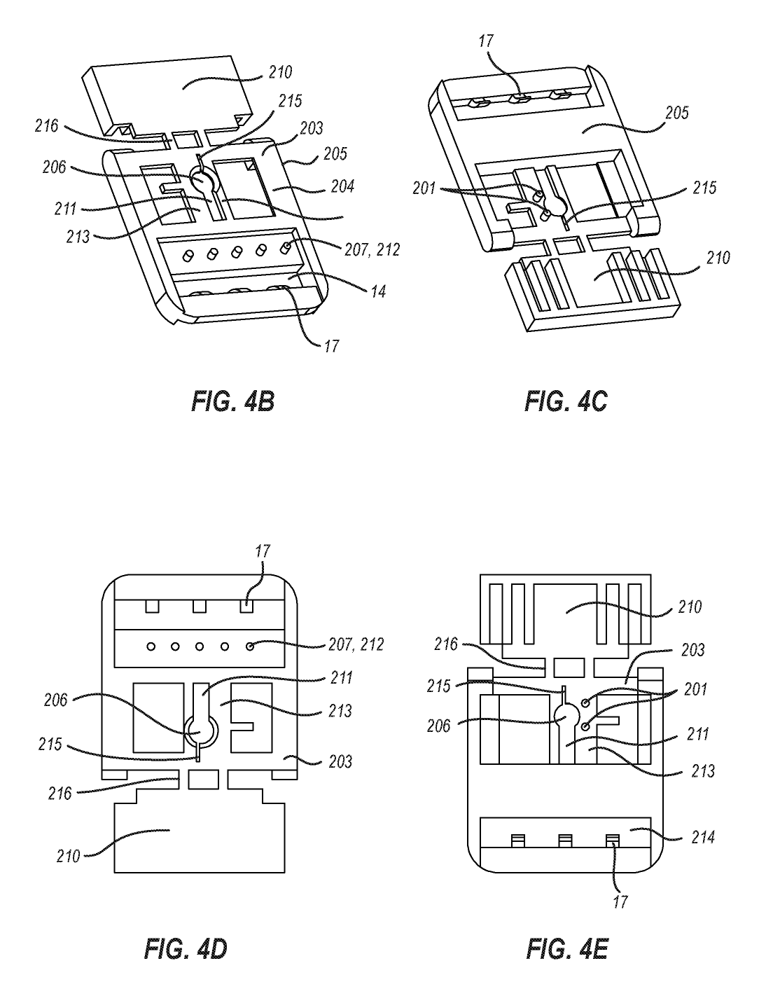

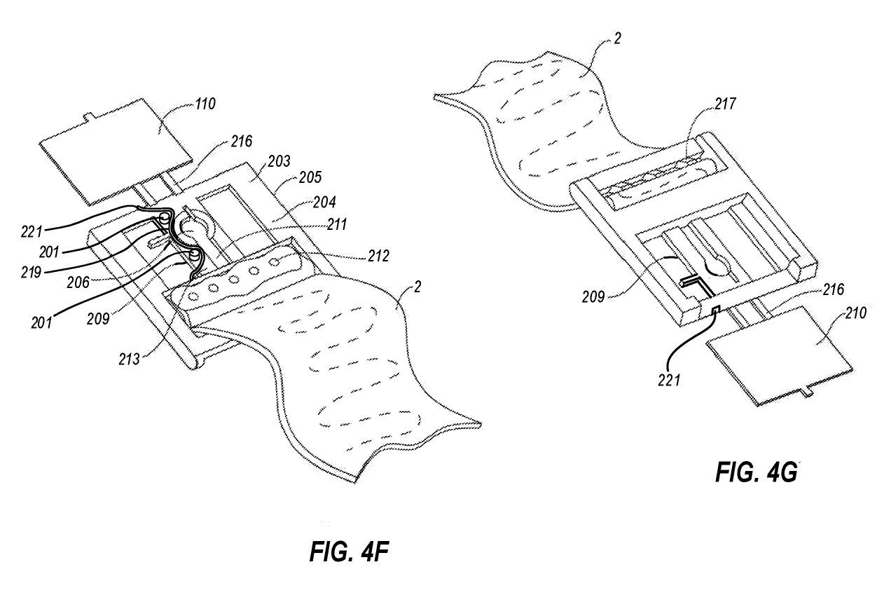

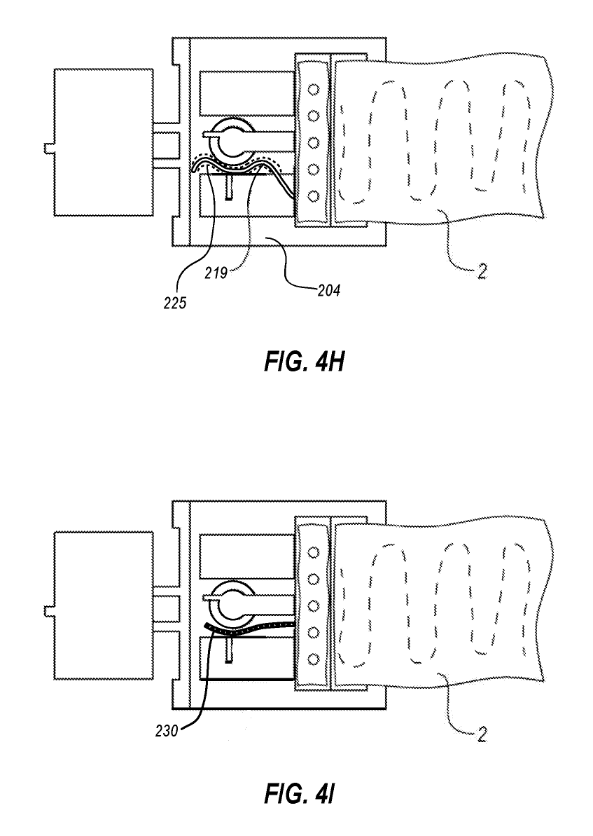

According another embodiment, as shown in FIGS. 4A to 4I, a contact portion 219 of an end portion of the belt electrode wire 209 is held on a rear side 205 of the frame 203 (the side of the frame 203 facing toward the biometric device). According to this embodiment, the contact portion 219 of electrode wire end 209 extends substantially along the rear side 205 of second member 213. Accordingly, when the male snap portion is inserted and snapped into hole 206, the contact portion 219 of the electrode wire end 209 is forced into contact with a surface of the male snap foundation 161, or a portion of the lateral surface of the protrusion shaft 167, or both. According to this embodiment, a reliable and repeatable electrical connection between the contact portion 219 of the electrode wire end 209 and the conductive male snap portion can be secured upon inserting and fastening the male snap portion 151 into receiving hole 206.

As shown in FIG. 4B-4D, contact members 201 can extend from frame 203 in a direction substantially perpendicular to the electrode wire end 209. Contact members 201 may each have lateral surfaces 202 configured to hold the contact portion 219 in place near the receiving hole 206. Similarly, electrode wire end 209 maybe formed to have a looped portion 221 to provide additional wire and area to contact portion 219 to contact the surface of male snap foundation 161.

In addition to, or alternatively to, the contact members 201, the contact portion 219 of the electrode wire 209 may be held or secured in place by an adhesive 225 applied to a front side 204 of second member 213, as shown in FIG. 4H. Or alternatively, or in addition to the contact members 201 or an applied adhesive, a groove or recessed portion 230 may be formed in a surface of first or second member, the groove or recessed portion 230 (FIG. 4I) being arranged to at least partially hold or secure the contact portion 219 of electrode wire 209 in place, at the front side 204, or alternatively at rear side 205 of the frame 203 in sufficiently close proximity to receiving hole 206 to make contact with the male protrusion 151 when inserted into receiving hole 206. First member 208 and second member 213 thus provide a resilient force not only to the male protrusion 151 when it penetrates through receiving hole 206, but first member 208 and second member 213 may also provide a contact force to push the contact portion 219 into a secure and reliable connection with a surface of the male snap foundation 161, or a portion of the lateral surface of the protrusion shaft 167, or both.

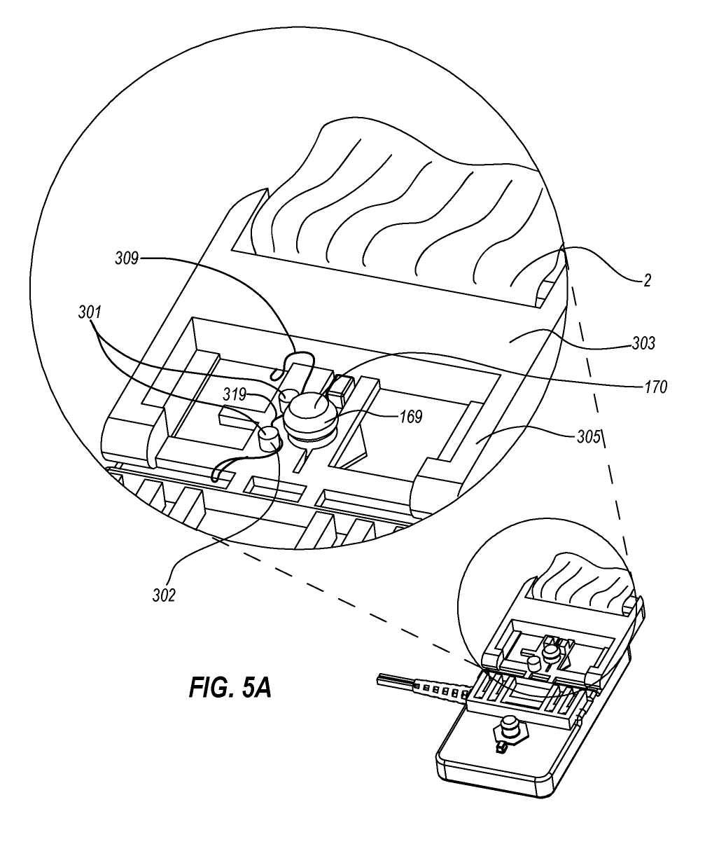

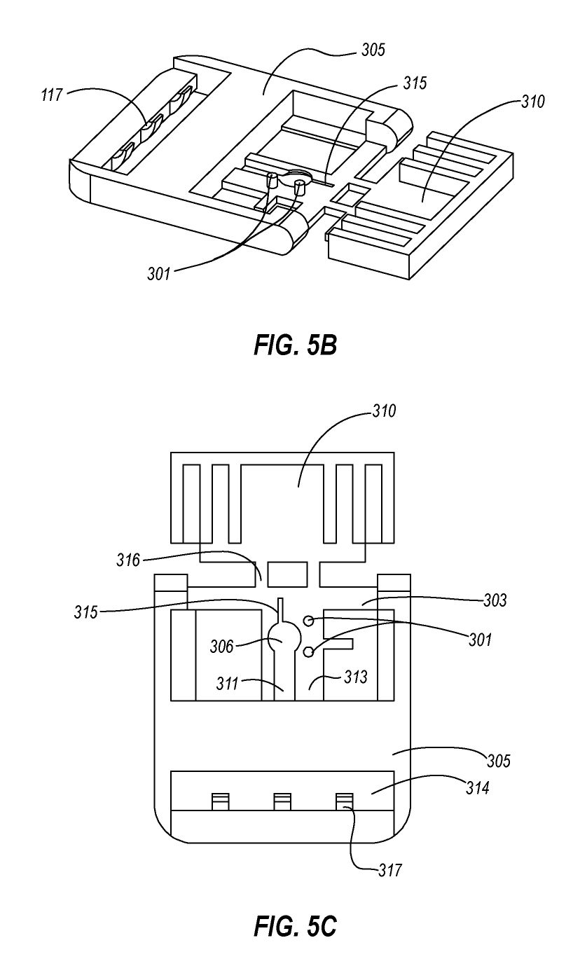

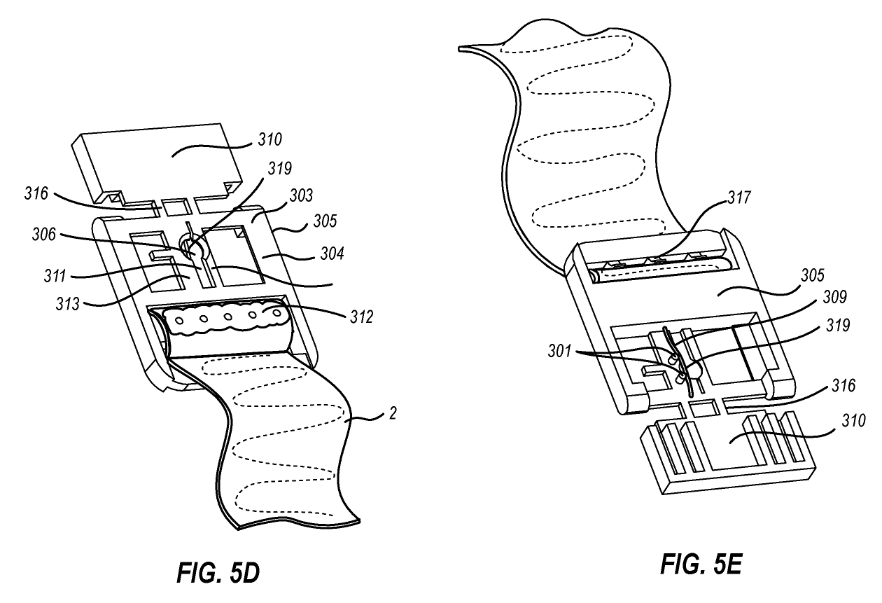

According to another embodiment, as shown in FIGS. 5A to 5E, a contact portion 319 of electrode wire 309 is held in place on a rear side 305 of frame 303, such that when the male protrusion 151 penetrates through receiving hole 306, the contact portion 319 is forced to the side of the hole 306 such that at least a portion of lateral surface 167 of protrusion shaft 166, a lateral surface 177 of bulged portion 175, a surface of top bevel 169, and/or a surface of underside bevel 168 is forced into contact with contact portion 319. Contact portions 319 may also be held in position by structural support from an inside surface of shield 310. According to the embodiment of FIG. 5A, contact members 301 having lateral surfaces 302 may be provided to hold the contact portion 319 in place and exert a lateral force upon contact portion 319 when the male snap portion 151 penetrates hole 306. Contact members 301 may be formed of various shapes so long as they provide the lateral support and force. As shown in the embodiment of FIG. 5A, the contact portions 319 may have a circular cross-sectional shape, but other shapes may also be used, such as square, rectangular, semi-circular, or a shape having a lateral surface 302 with a concave surface area. There may be a single contact members, or as shown in FIG. 5A, two or more contact members 301 may be provided.

In addition to, or alternatively to, the contact member(s) 301, the contact portion 319 of the electrode wire 309 may be held or secured in place by an adhesive applied to first or second member. Or alternatively, or in addition to the contact members 301 or an applied adhesive, a groove or recessed portion may be formed in a surface of first or second member 308, 313, the groove or recessed portion being arranged to at least partially hold or secure the contact portion adjacent to the rear side of the receiving hole 306. Alternatively, a contact member, adhesive, or groove, or other means, may be provided on an inside surface of shield 310 or another lid structure to maintain the contact portion 319 in lateral contact with the male snap portion 151 when inserted into hole 306.

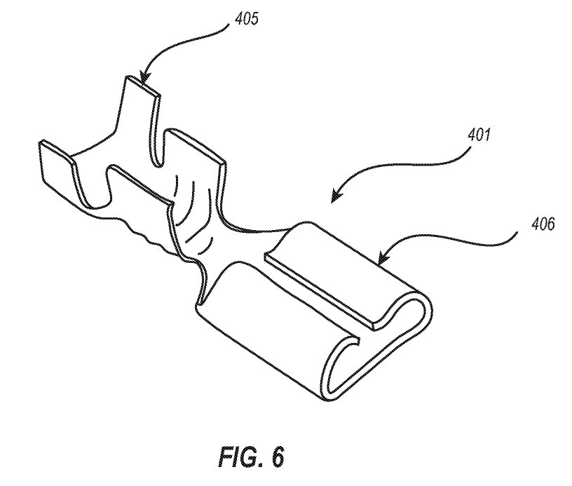

In other embodiments, as shown in FIG. 6, a conductive connector or bridging conductor may be used to contact a surface of the male snap portion. Such a conductive connector may include an electrical crimp shoe 401, including a wire connector 405 and a contact portion 406, the wire connector 405 being configured to be connected (such as by crimping) to an end portion of the electrode wire of the belt. The contact portion 406 may be arranged with the belt connector frame in such a way as way to contact a surface of a male snap portion 151. A belt connector according to such an embodiment may provide a reliable and easily repeatable connection between a biometric device and a wire belt electrode.

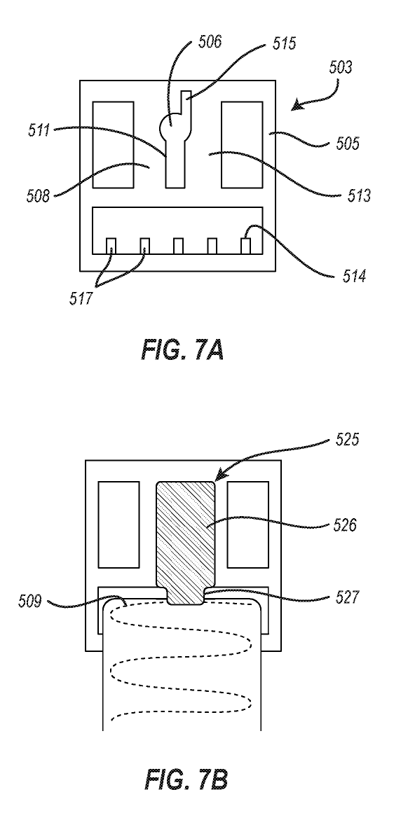

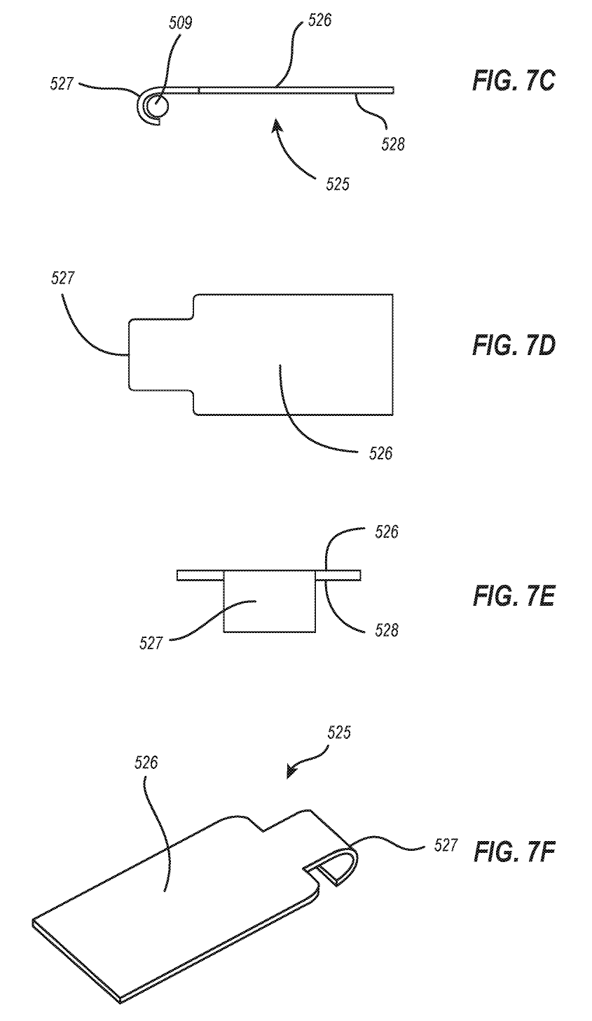

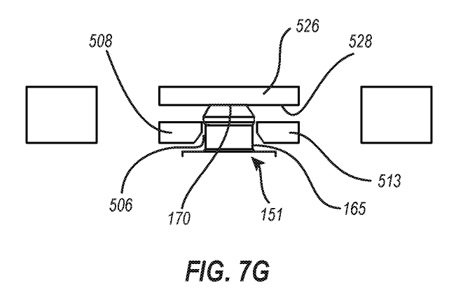

According to an embodiment, as shown in FIGS. 7A to 7G, a frame 503 may be provided having a rear side 505. The frame may include a first member 508 and a second member 513 that define a receiving hole 506. The first member 508 and second member 513 may further define a first slot 511, and may additionally define a second slot 515. The frame 503 may further include a belt slot 514 with teeth 517 configured to attach the frame 503 to a belt 2, as shown in FIG. 7B.

A conductive connector 525 may be provided and coupled at least partially to the surface of the frame 503 at the rear side 505 of the frame (opposite from the biometric device connector). Conductive connector 525 may include a contact plate 526 that covers, at least partially or entirely, the receiving hole 506. Conductive connector 525 may further include a wire connector 527, which may be a wire crimp configured to be crimped onto an end portion 509 or an electrode wire end of belt 2. As shown in FIG. 7C, contact plate may have a contact surface 528 configured to at least partially contact a top surface 170 or a portion of top bevel 169, or both, of the male snap portion 151 when the male snap portion is inserted and fastened within receiving hole 506, as shown in the cross-sectional view of FIG. 7G. When inserted into receiving hole 506, first member 508 and second member 513 may provide a radial flexibility to the receiving hole 506, which causes the male snap portion 151 to be securely held within receiving hole 506.

Additionally, although not shown, similar to other embodiments, frame 503 may include a shield or lid portion attached to the frame body by one or more flexible hinges. A shield or lid portion may provide additional support to a surface of contact plate 526 opposite from the contact surface 528, to provide a more secure electrical connection between the contact plate 526 and the tope surface 170 or a portion of top bevel 169 of male snap protrusion 151.

Conductive connector 525 may be coupled to frame 503 by an adhesive. Conductive connector 525 may also be coupled to frame 503 through a crimping mechanism. Contact plate 526 may also be bent to have a concavity facing away from the frame 503, such as to provide a spring force in an opposite direction to the insertion direction of the male snap portion 151 into the hole 506. Such a spring force may provide a secure and solid contact of the contact plate 526 against the top portions of the male snap portion 151 during movement.

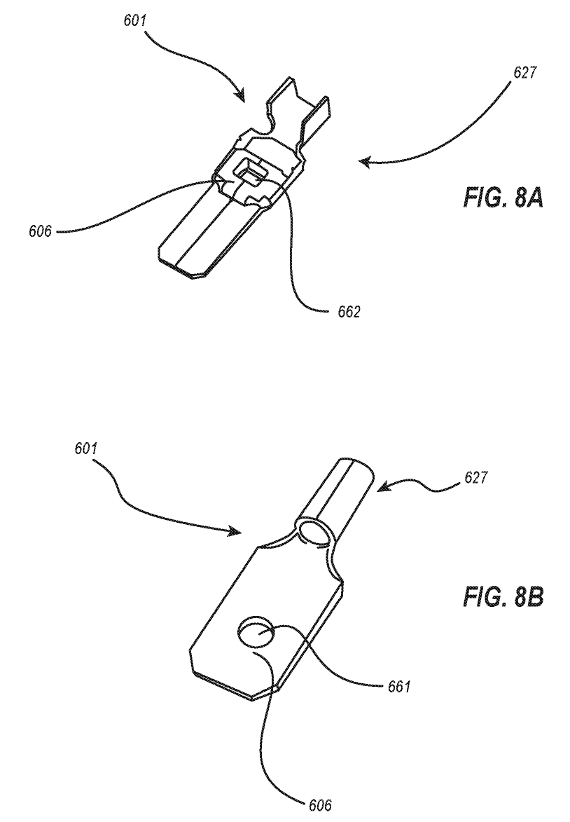

As shown in the embodiments of FIGS. 8A and 8B, the conductive connector 601 may include a contact portion 606 and a wire connector 627. In the embodiment of FIG. 8A, the contact portion 606 may include a rectangular cut-out 662 configured to couple or contact a top portion (top surface 170 or a portion of top bevel 169) of the male snap portion 151. In the embodiment of FIG. 8A, the rectangular cut-out 662 may be configured to mate with a male snap portion having a rectangular or square cross-sectional area or rectangular or square top surface. In the embodiment of FIG. 8B, a circular cut-out 661 is provide configured to mate with a male snap portion having a circular cross-sectional area, or a circular top surface. In another embodiment, the conductive connector may be forked-shaped, with a base portion and two arms that extend parallel to each other from the base portion. The fork-shaped conductive connector may be configured to be fit on a rear side 605 of the frame 603, and may be arranged between the frame 603 and a lid or shield. With such a fork-shaped conductive connector, an internal portion of the arms may be forced into contact with a top portion of the male snap protrusion 151.

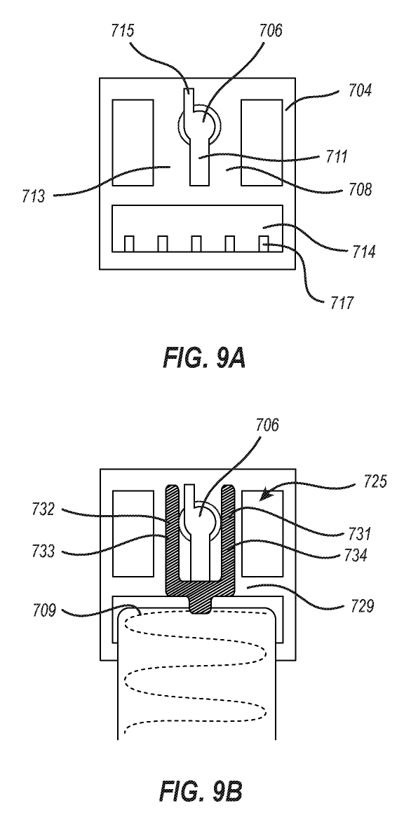

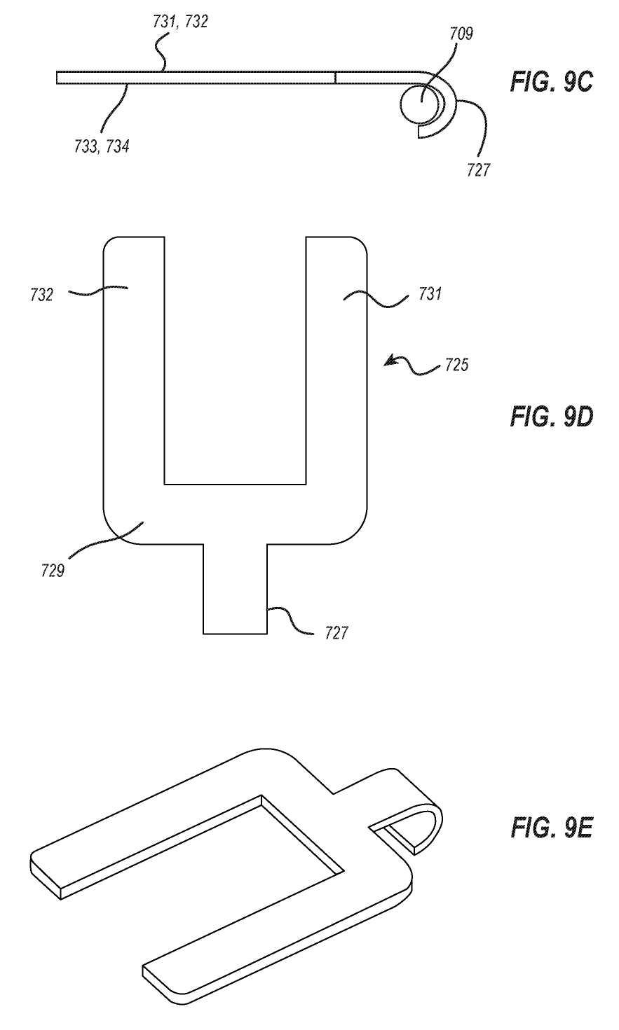

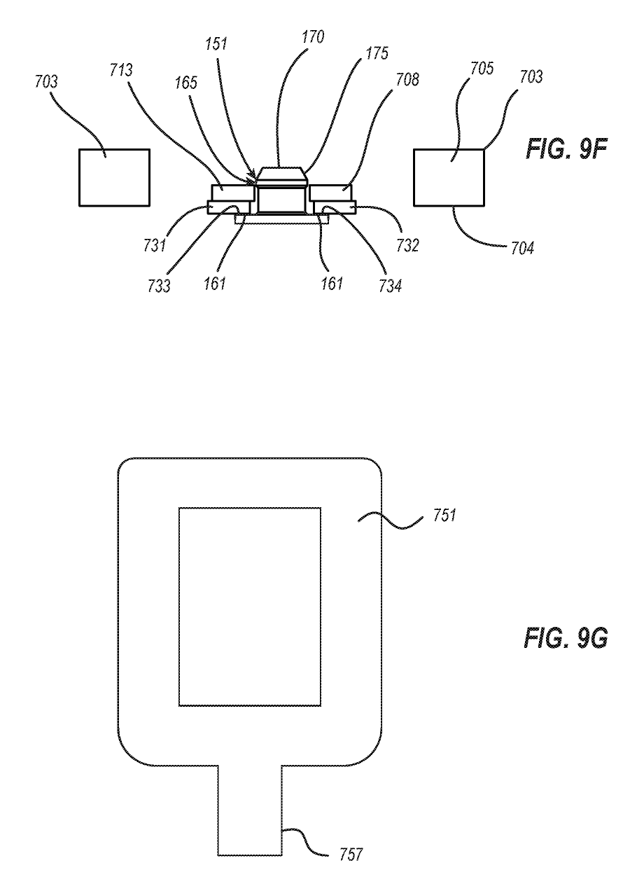

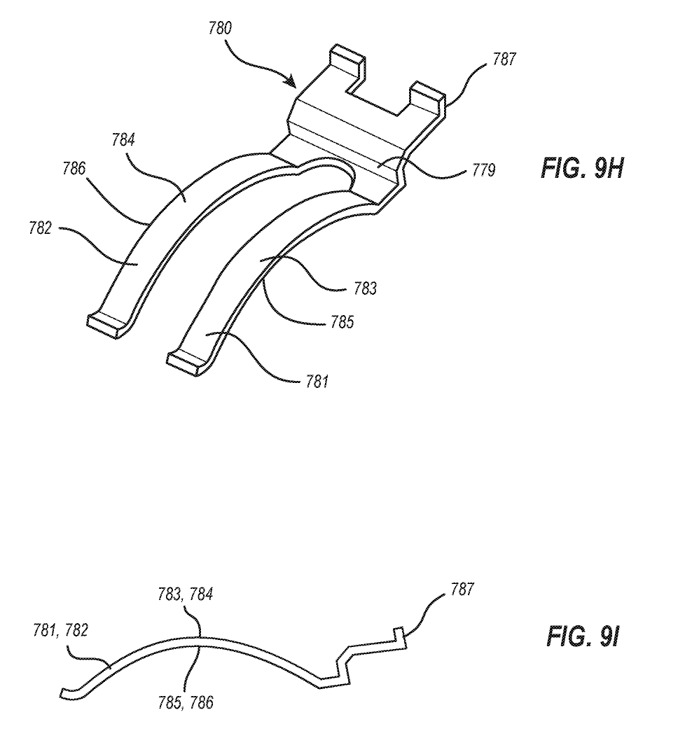

According to another embodiment, as shown in FIGS. 9A to 9F, a frame 703 having a front side 704 and a rear side 705, may be configured to be coupled to a fork-shaped conductive connector 725 on the front side 704 of frame 703. The fork-shaped conductive connector 725 may include a base portion 729, a first arm 731, and a second arm 732. The first arm 731 may be substantially parallel to the second arm 732. The conductive connector 725 may further include a wire connector 727 (such as wire crimp), configured to connect the conductive connector, both physically and electrically, to the electrode wire end 709 of the electrode belt. Each, or at least one of, first arm 731 and/or second arm 732 may have a contact surface 733, 734, configured to contact a surface of male snap foundation 161, a lateral surface 167 of protrusion shaft 165, or both.

Thus, as shown in the cross-sectional view of FIG. 9F, first arm 731 is provide between first member 708 and a portion of male snap foundation 161, and/or second arm 732 is provided between second member 713 another portion of male snap foundation 161, when the male snap portion 151 is inserted through hole 706. The underside bevel 168, in conjunction with the radial flexibility of the first member 708 and 713 providing flexibility transvers to their longitudinal axis, thus forcing the first arm 731 and the second arm 732 into secure contact with the male snap foundation 161.

According to another embodiment, as shown in FIG. 9G, the conductive connector may be a rectangular ring-shaped conductive connector 751, with a wire connector 757. The ring-shaped conductive connector 751 may be configured to be placed on a front side 704 of the frame 703, similar to the fork-shaped conductive connector 725, to be placed into secure physical and electrical contact with the male snap foundation 161.

In another embodiment, shown in FIGS. 9H and 9I, the first arm 781 and second arm 782 of fork-shaped conductive connector 780 may extend from base portion 779 and may be bent to form a concave surface facing toward the frame 703, such that a spring force is provided by the first arm 781 and second arm 782. A secure contact may be provided between contact surfaces 783 and 784 (of first arm 781 and second arm 782, respectively) and the male snap foundation 161, when the male snap portion 151 is inserted into the receiving hole. Conductive connector 780 may include a wire connector 787 (such as a crimp) to physically and electrically connect the conductive connector to an end portion of a wire electrode of the electrode belt.

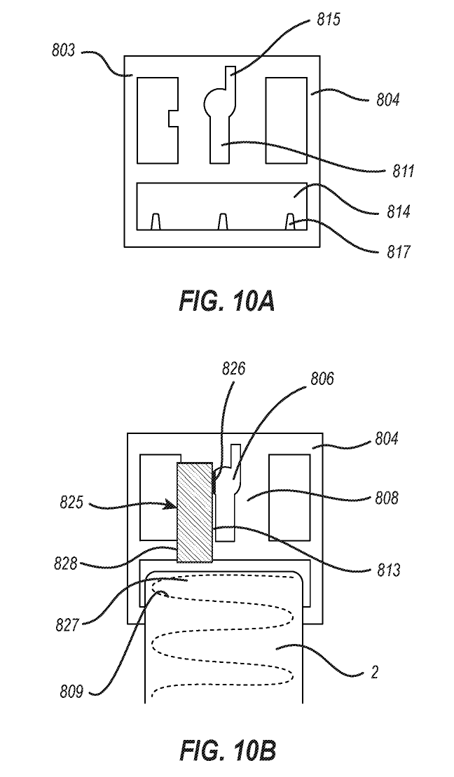

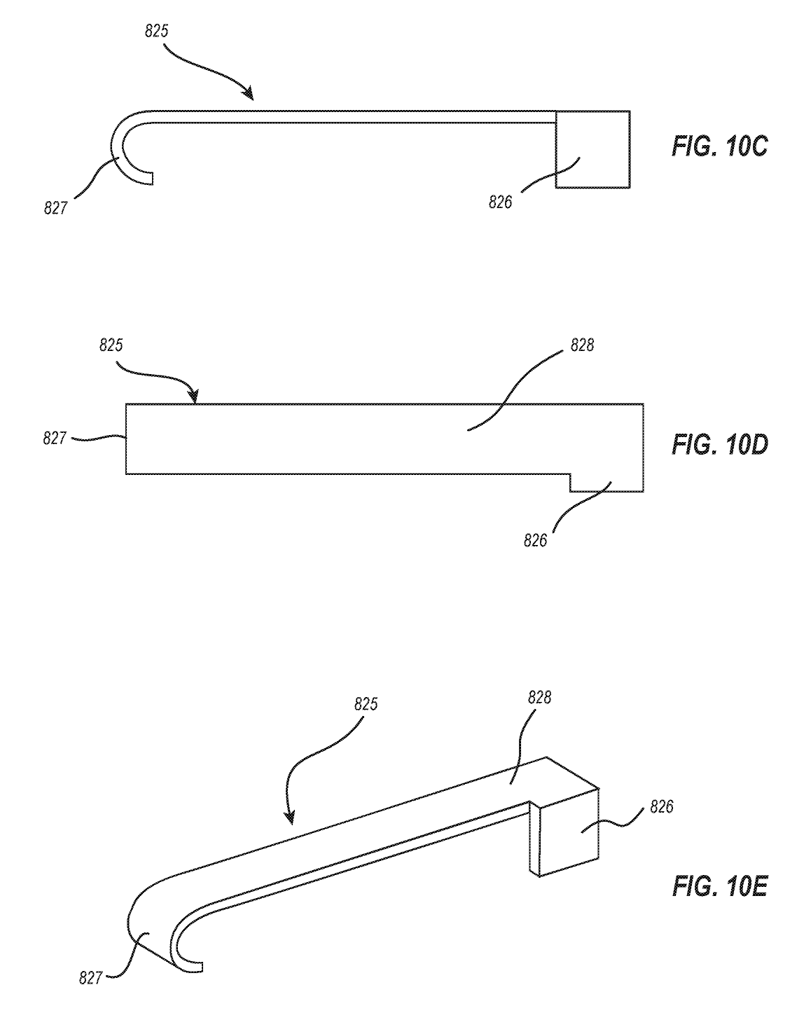

As shown in FIGS. 10A to 10E, in another embodiment, a conductive connector 825 is provided with a contact portion 826 configured to at least be partially inserted into receiving hole 806 of frame 803. In this embodiment, frame 803 includes first member 808 and second member 813 that define lateral sides of receiving hole 806. First member 808 and second member 813 may also define first slot 811 and second slot 815. Frame 803 has a front side 804 (configured to face toward the biometric device) and a rear side 805. Frame 803 is configured to be coupled to conductive connector 825.

Conductive connector includes body portion 829 extending from the wire connector 827 to the contact portion 826. Contact portion 826 may be arranged (such as by bending) to extend in a plane perpendicular or nearly perpendicular to the direction in which the body portion 829 extends. Contact portion 826, which may be shaped as a flag, thus may be inserted and attached into receiving hole 806 such that when male snap portion 151 is inserted into hole 806, a contact surface of contact portion 826 may be brought into physical and electrical contact with a surface of male snap portion 151. Radial flexibility of at least one of the first member 808 and second member 813 may provide a lateral force substantially perpendicular to the longitudinal axis of the first member 808 or second member 813 as to force the contact portion securely against a lateral surface of male snap portion 151.

Conductive connector 825 may be coupled to frame 803 by a crimping of the conductive connector to the frame, an adhesive, tension fitting, heat fitting, shrink fitting, a fastener, or other means of connection.

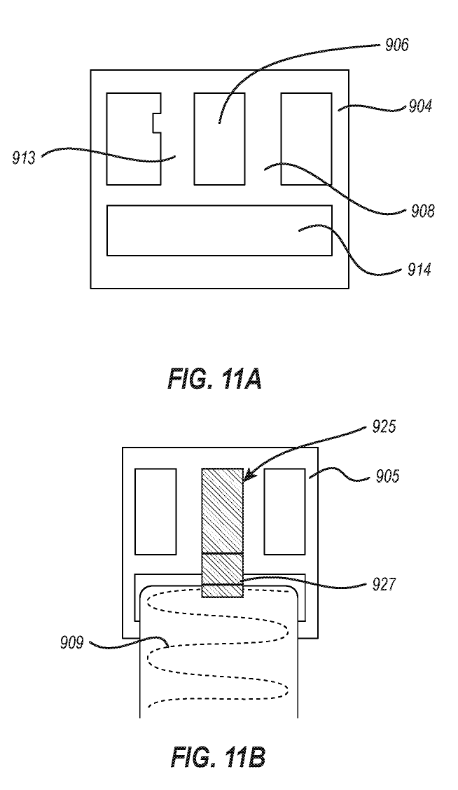

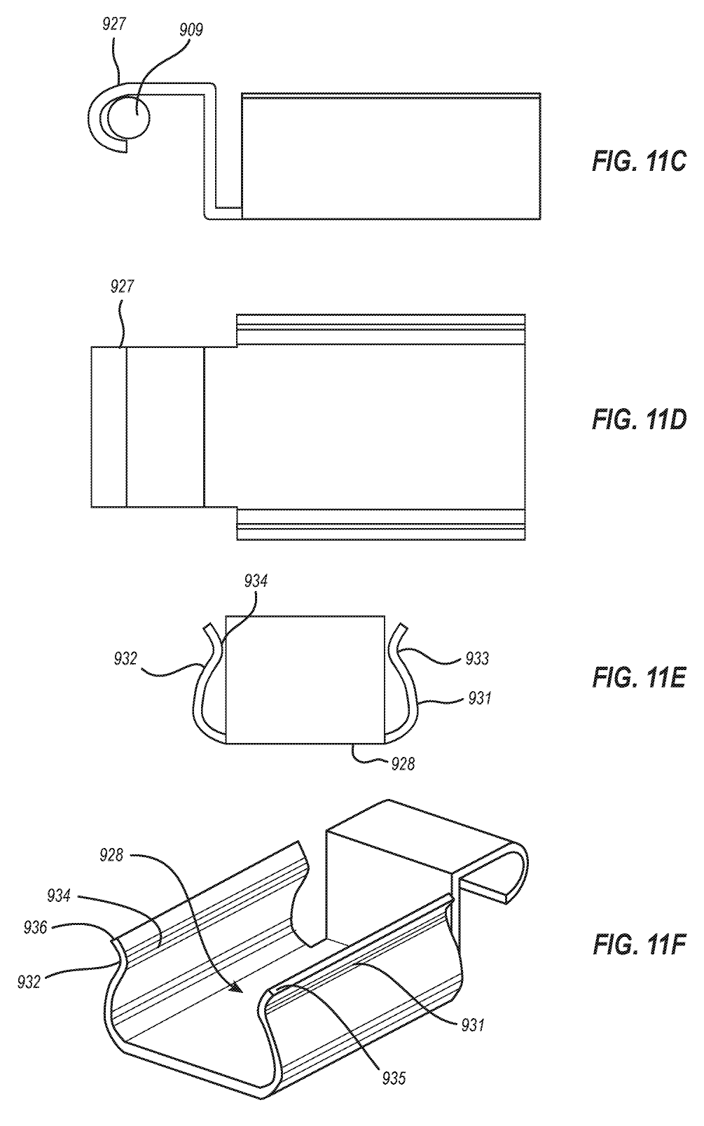

In another embodiment, as shown in FIGS. 11A to 11F, a U-shaped conductive connector 925 is provided. According to this embodiment, as shown in FIG. 11A, a frame 903 includes a first member 908 and second member 913 provided adjacently to and define, at least in part, receiving hole 906. Receiving hole 906 may be rectangular in shape, so as to receive and mate with a U-shaped conductive connector 925. In this embodiment, the U-shaped conductive connector 925 is inserted into frame 903 and wire connector 927 is connected to electrode wire 909, such as by crimping, soldering, or fastening. U-shaped conductive connector 925 is arranged with the opening of the U-shape, or the concavity of the U-shape to be pointed toward the front side 904 of the frame 903 such that the male snap portion 151 may be inserted into the opening formed by the U-shaped conductive connector 925. As seen in a cross sectional view (FIG. 11E), U-shaped conductive connector has first side portion 931 and an opposing second side portion 932, each extending from base portion 928. The first side portion 931 may have first contact surface 933 on the inside of first contact surface 931 and second contact surface 934 may have on the inside surface of second side portion 932. Through the lateral force provided by the first member 908 and/or the lateral force provided by the second member 913, the first contact surface 931 and second contact surface 934 are forced into secure contact with a lateral surface of male snap portion 151.

Additionally, a restorative spring force of the first side portion 931 and second side portion 932 may provide a lateral force to hold first contact surface 931 and second contact surface 934 in contact with a lateral surface of male snap portion 151. On a side of the conductive connector 925 opposite from the direction in which the first and second side portions extend, a shield (not shown) or lid attached to the rear side of the frame 903 may give additional support in the direction toward the biometric device. Additionally, respective lip portions 935, 936, of first and second side portions 931, 932, may extend onto a surface of front side 905 of the frame 903, so as to maintain the conductive connector 925, particularly when male snap portion 151 is inserted within the U-shaped conductive connector and hole 906.

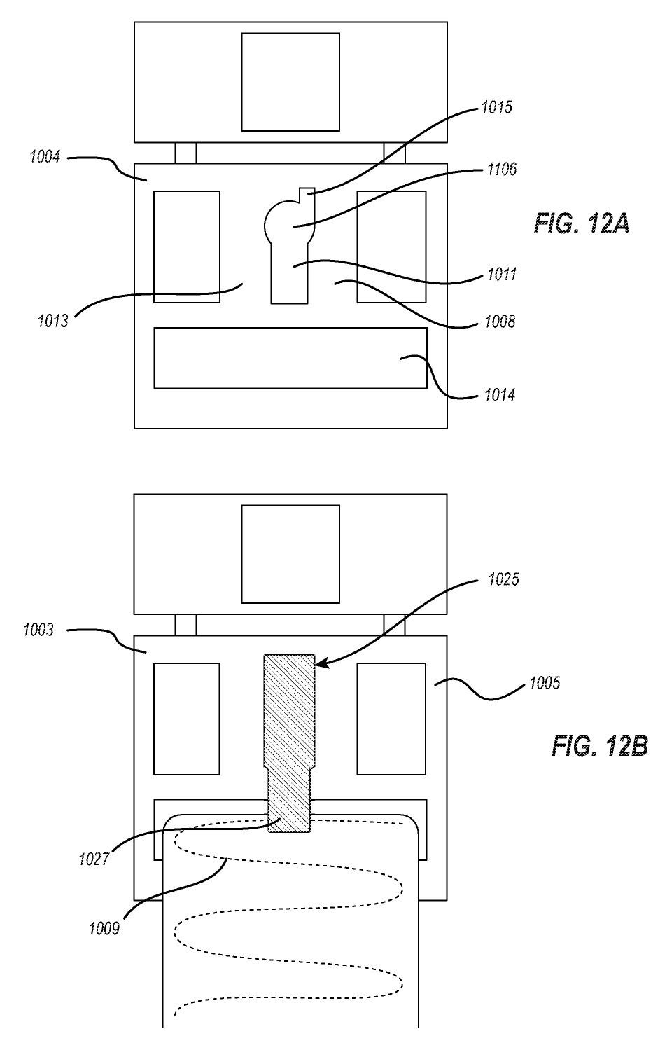

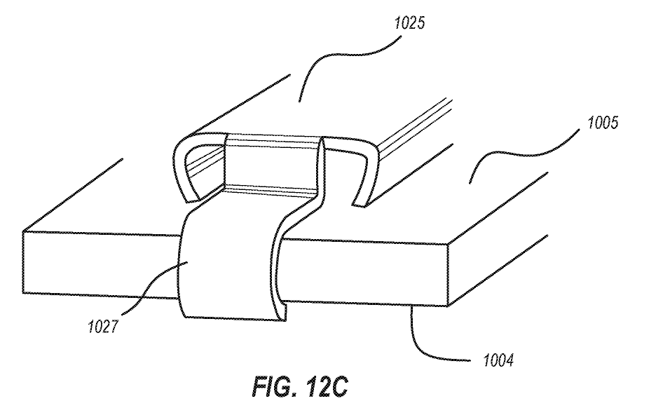

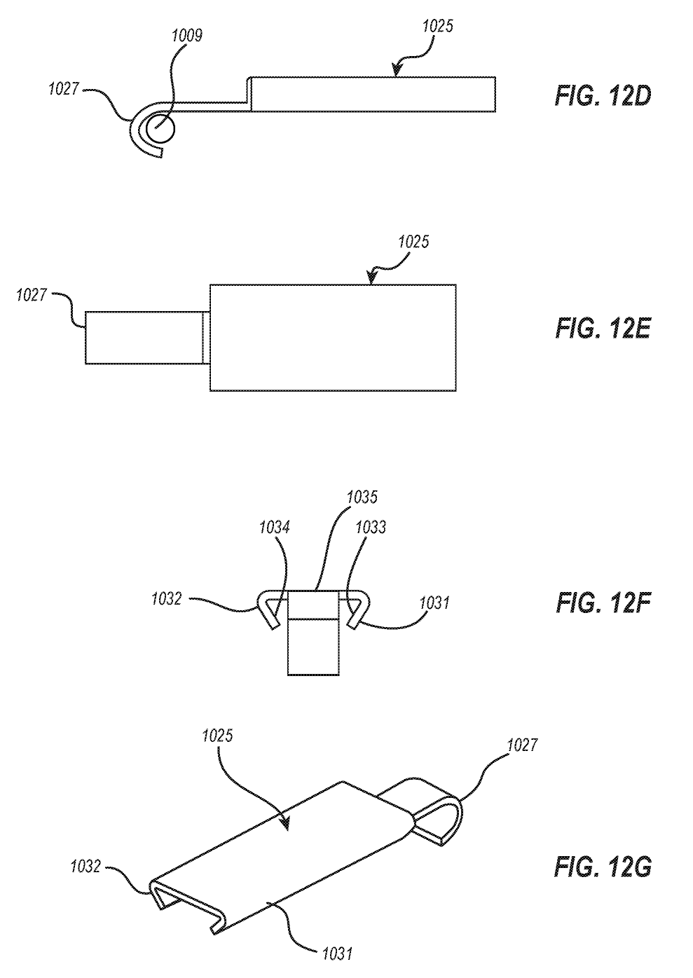

In another embodiment, as shown in FIGS. 12A to 12G, a frame 1003 has a front side 1004 and a rear side 1005. The frame may include a first member 1008 and a second member 1013, which define at least partially, a receiving hole 1006 with radial flexibility. First member 1008 and 1013 may further define first slot 1011 and second slot 1015, which may contribute to the radial flexibility provided at receiving hole 1006. A top spring conductive connector 1025 is provided having a wire connector 1027, a first contact member 1031, and a second contact member 1032. The top spring conductive connector 1025 may be arranged on a rear side 1005 of frame 1003, such that when a male snap portion 151 is inserted within receiving hole 1006, lateral surfaces of male snap portion are brought and held in contact with inner contact surfaces of conductive connector 1025, including first contact surface 1033 of first contact member 1031 and first contact surface of second contact member 1032. Additionally, electrical contact can be made between an inner surface of the base portion 1035 of the conductive connector and a top surface 170 of the male snap portion 151. The male snap portion 151 may then be securely held fastened to the belt connector due to the radial flexibility provided by first and second elongated members 1008 and 1013, and may also be held in place by the spring force provided by the first contact member 1031 and the second contact member 1032. Conductive connector 1025 may be coupled to frame 1003 by a crimping of the conductive connector 1025 to the frame, an adhesive, tension fitting, heat fitting, shrink fitting, a fastener, or other means of connection.

Wire connector 1027 may provide an electrical and physical connection between the conductive connector 1025 and the electrode wire end 1009.

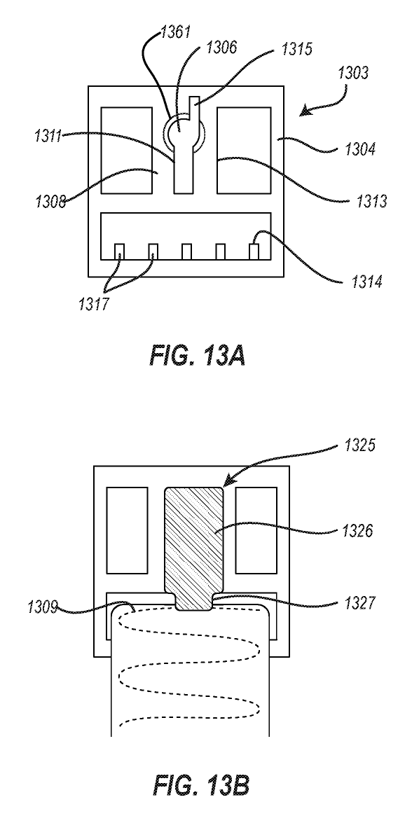

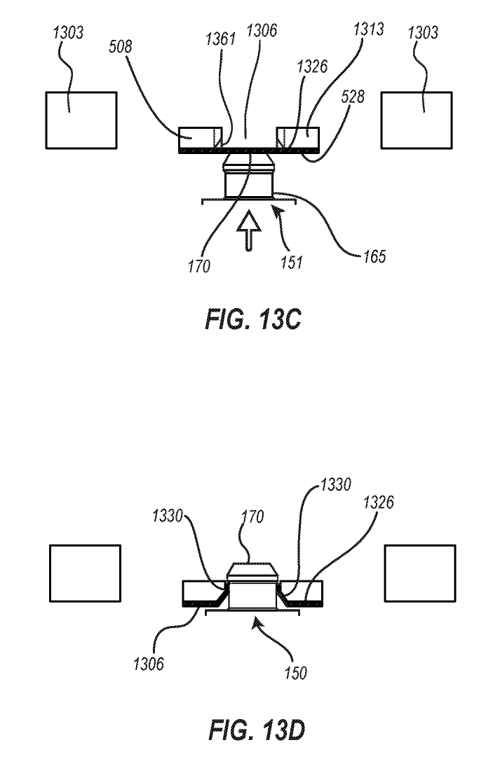

In another embodiment, as shown in FIGS. 13A to 13D, a frame 1303 has a front side 1304 and a rear side 1305. The frame may include a first member 1308 and a second member 1313, which define at least partially, a receiving hole 1306 with radial flexibility. First member 1308 and 1313 may further define one or more slots extending from said receiving hole 1306, for example, first slot 1311 and second slot 1315, as shown in FIG. 13A. Said slots may contribute to the radial flexibility provided at receiving hole 1306. Said receiving hole 1306 may have a bevelled edge 1361 on front side 1304 to more easily receive and guide a male snap 151 fastener into hole 1306. When inserted into receiving hole 1306, first member 1308 and second member 1313 may provide a radial flexibility to the receiving hole 1306, which causes the male snap portion 151 to be securely held within receiving hole 1306. Similar to other embodiments, the frame 1303 may further include a belt slot 1314 with teeth 1317 configured to attach the frame 1303 to a belt 2, as shown in FIG. 13B.

As shown in FIG. 13B, a conductive connector 1325 may be provided the front side 1304 of the frame 1303. The conductive connector may be a conductive foil configured to be easily penetrable by male snap portion 151 upon insertion of the male snap portion 151 into receiving hole 1306. The conductive foil may be a metal foil, for example, a copper foil. The metal foil may also be an aluminium foil, or some other malleable metal foil. The conductive foil may also be a polymer or plastic foil with a conductive material provided thereon, such as a plastic or polymer foil with an aluminium or copper layer formed therein. Conductive connector may include a wire connector 1327 configured to crimp to the wire 1309 at an end portion of the wire of the belt. The wire connector may also include a solder portion or electrical adhesive that electrically and physical connects the conductive connector to the wire 1309 of the belt.

As shown in FIG. 13B, the conductive connector 1325 include a surface contact portion 1326 configured to contact the snap portion 151 and allow penetration and tearing at least of a portion of the conductive connector 1325 upon penetration of the male snap portion 151 into the receiving hole 1306. Although not shown, perforations may be provided at surface contact portion 1326.

As shown in FIG. 13C, which shows a cross-sectional view of the embodiment of FIG. 13B, a top surface 170 of male snap portion 151 may be brought to contact the surface contact portion 1326 of the conductive connector 1325, and upon application of sufficient force (for example, manual force) the male snap portion 151 may be forced through surface contact portion 1326, for example, by tearing at least a portion of the conductive connector 1325, as shown in FIG. 13D, at surface contact portion 1326. Upon insertion of the male snap portion 151 into receiving hole 1306 through a portion of the conductive connector 1325, one or more folded portions 1330 of conductive connector 1325 may be forced through at least a portion of the receiving hole 1306 and forced by the radial force applied by members 1308 and 1313 into contact with a least a lateral portion of male snap portion 151. Further, a remaining portion of surface contact portion 1326 provides additional electrical contact and connection by contacting a surface of base portion 161 of the male snap electrode 151. Thus, a reliable electrical connection can be provided between the wire 1309 of the belt and the male snap portion 151, at reduced overall manufacturing costs.

In the embodiments above, radial flexibility of the receiving hole may be provided by the first member and the second member, which define at least partially the receiving hole and slots, such as a first slot and second slot, the second slot being arranged on a different side of the receiving hole from the first slot. But radial flexibility may be provided by different means. For example, a receiving hole may have radial flexibility due only to the material surrounding the receiving hole. For example, a frame may be formed entirely or partially from an elastic material, such a rubber or other elastic polymer. In another embodiment, a frame may include a portion, such as elongated members, formed of a molded and more firm plastic, while inner portion adjacent to the receiving hole, may be formed of a softer, more flexible and resilient material. Such a frame may be combined with the above-described configurations to provide a belt connector with a conductive connector or wire configured to securely contact a surface (either top, lateral, or foundation) of a corresponding male snap portion.

Additionally, in many of the above-described embodiments, the male snap portion may have a circular cross-sectional area in the top plan view. However, the male snap portion may have other shapes in the cross-sectional area in the top plan view, such as a rectangular, square, elliptical, triangular, or an asymmetric shape.

In another aspect, the present disclosure sets forth a process for making an electrode belt with biometric belt connectors, comprising:

placing an end of a flexible electrode belt with an incorporated wire onto a belt connector as defined above, in the suitable direction in which it is to be fastened onto the connector, such that a portion of the belt end extends beyond said ridge member or row of pins,

pressing a heat element ultrasonic hot body or other means of heat transfer onto said belt and ridge member, and through the action of the heat, shearing an end piece of the belt but leaving intact the incorporated wire, thus revealing an end of said wire, through the action of heat from said heat element, fastening by heat melting said belt to said ridge, and

fastening the wire end to a member adjacent to the hole of the connector frame, such that said end is in electrical contact with said hole and comes in electrical contact with a conducting male snap fastener inserted in said hole.

The process may further comprises enclosing the connector frame with the fastened belt end and connected wire with a cover such as suitably a cover as described above. The cover may include a shield coupled to the connector frame by flexible hinges.

According to another embodiment of the process for making an electrode belt with a biometric belt connector, a frame may be provided, the frame the frame having a receiving hole with radial flexibility. A conductive connector may be coupled to the frame, the conductive connector providing a secure and reliable electrical connection between the male snap portion that the connector is configured to receive and an electrode wide of the fastened belt.

The belt connector of the present disclosure may include the following features, or the various combinations of features as described below.

1. An electrode belt connector for electrically connecting a conductor of an electrode belt to a first snap portion of a snap connector electrode configured to be connected to a biometric device, the electrode belt connector comprising:
a frame defining a corresponding second snap portion, configured to mate with the first snap portion of the snap connector electrode; and
an connecting portion configured to electrically connect the conductor of the electrode belt to the first snap portion of the snap connector.
2. Any one of the above or below connectors wherein the first snap portion is a male snap portion.
3. Any one of the above or below connectors wherein the second snap portion is a receiving hole.
4. Any one of the above or below connectors wherein the second snap portion is a male snap portion.
5. Any one of the above or below connectors wherein the first snap portion is a receiving hole.
6. Any one of the above or below connectors wherein the electrode belt is a respiratory inductive plethysmography belt.
7. Any one of the above or below connectors wherein the male snap portion includes a male snap foundation.
8. Any one of the above or below connectors wherein the male snap portion includes a male snap protrusion extending substantially perpendicularly from the male snap foundation.
9. Any one of the above or below connectors wherein the male snap portion is electrically connected to the internal electrical leads of the biometric device.
10. Any one of the above or below connectors wherein the male snap protrusion includes a cylindrically shaped protrusion shaft that extends perpendicular or nearly perpendicularly from the surface of male snap foundation.
11. Any one of the above or below connectors wherein the protrusion shaft has a lateral.
12. Any one of the above or below connectors wherein on a distal end of the protrusion shaft, the cross-sectional diameter of the male snap protrusion enlarges at a underside bevel forming a bulged portion, the bulged portion being formed at the distal end of the male snap protrusion.
13. Any one of the above or below connectors wherein the underside bevel extends to a lateral surface of the bulged portion.
14. Any one of the above or below connectors wherein the lateral surface has a cylindrical shape with a cross-sectional diameter greater than the protrusion shaft.
15. Any one of the above or below connectors wherein the bulged portion has a top bevel where the cross-sectional diameter reduces.
16. Any one of the above or below connectors wherein a top surface is formed adjacent to the upper edge of the top bevel.
17. Any one of the above or below connectors wherein male snap portion is made of an electrically conductive material, including, brass, copper, aluminium, steel, gold, or other metals or metal alloys, or even non-metal electrically conducting materials.
18. Any one of the above or below connectors wherein the biometric belt connector is electrically connected to an electrode belt.
19. Any one of the above or below connectors wherein the belt connector comprises a frame having a front side and a rear side.
20. Any one of the above or below connectors wherein the frame defines a shaped circular or semi-circular hole 6 with radial flexibility to function as a female snap button fastener.
21. Any one of the above or below connectors wherein the frame is a molded frame.
22. Any one of the above or below connectors wherein the frame is made from a molded plastic material.
23. Any one of the above or below connectors wherein the frame includes fasteners, which comprise a ridge member to physically secure the belt to the frame.
24. Any one of the above or below connectors wherein the ridge member includes a series of butts or pins 7 which are provided transverse to the belt direction and to which the belt end can be fastened onto with heat melting or gluing.
25. Any one of the above or below connectors wherein the frame include two members, first member and second member, that extend adjacently to the hole.
26. Any one of the above or below connectors wherein first member and second member extend parallelly to each other.
27. Any one of the above or below connectors wherein first member is arranged at an angle to second member other than extending parallelly.
28. Any one of the above or below connectors wherein the two members form a first slot that extends from the hole.
29. Any one of the above or below connectors wherein first member and second member also form a second slot.
30. Any one of the above or below connectors wherein the second slot is formed across the hole from the first slot.
31. Any one of the above or below connectors wherein the two slots extend across from each other in the belt direction.
32. Any one of the above or below connectors wherein the one or more slots are formed or defined by at least one of the first or second elongated members.
33. Any one of the above or below connectors wherein members have flexibility transverse to their longitudinal axis (e.g. by being sufficiently thin), thus imparting flexibility to the width of the hole.
34. Any one of the above or below connectors wherein the hole is between two the elongated members, where one or both have sufficient and suitable flexibility to provide a snap fastener hole with suitable fastening strength.
35. Any one of the above or below connectors wherein either one of first member or second member, formed adjacent to said snap fastener receiving hole, engages an electrode wire coming from the belt end.
36. Any one of the above or below connectors wherein the wire is pushed into electrical contact with said receiving hole, either by extending into the hole, through the hole a single time or extending through the hole multiple times, for example, by being wrapped around one or both of the first member or the second member, or coming in electrical contact with the hole, e.g., through a bridging conductor or conductive connector.
37. Any one of the above or below connectors wherein the elongated members and the slots are formed to provide the hole with sufficient flexibility (i.e. elasticity in the width of the hole) to function as a female snap fastener.
38. Any one of the above or below connectors wherein the first or second member also functions to engage an electrode wire end from the belt end electrically connecting the belt with the hole and which comes in electrical contact with a conducting male snap fastener inserted in said hole.
39. Any one of the above or below connectors wherein the connector further comprises a belt slot with teeth members or pins, through which slot a loop of said belt can be inserted such that it is held by the teeth/pins when pulled back, to adjust the length of the belt.
40. Any one of the above or below connectors wherein the connector further comprises a shield member and/or a lid which are be molded in one piece with the frame and joined to the frame with foldable hinges such that the shield member or lid can be folded over to cover the rear side of the hole and/or wire end.
41. Any one of the above or below connectors wherein the wire end is crimped onto said member such that the crimping tubing fixes the wire and conducts and connects electrically the wire to the receiving hole, such that thus the wire and the belt is in electrical contact with a conducting male snap fastener inserted in said receiving hole.
42. Any one of the above or below connectors wherein the slot functions to provide an additional opening for a mating male projection on the biometric device such that it is assured that the device cannot be (or is unlikely to be) incorrectly fastened, and the device will not fit any generic non-proprietary belts having connectors with female fasteners but without the correctly shaped and placed extended hole.
43. Any one of the above or below connectors wherein the connector frame has a separate further hole, not joined to the main fastener and electrical connection hole, where the further hole is configured to mate with a corresponding male projection on said biometric device.
44. Any one of the above or below connectors wherein the biometric device has a female hole for mating with a corresponding male projection on the belt connector.
45. Any one of the above or below connectors wherein the connector frame further comprises a fastener to fasten the frame to a belt end of an electrode belt.
46. Any one of the above or below connectors wherein the fastener comprises a slot with a row of teeth, pins or hooks, transverse to the belt direction, to engage a belt end.
47. Any one of the above or below connectors wherein the belt slot provides for insertion of a loop of the belt through the belt slot to be held securely by the row of teeth, pins, or hooks, such that the belt length is adjusted and fixed, so that a user can later re-adjust the length.
48. Any one of the above or below connectors wherein the fastener comprises a ridge member, which is a flat or sharp elongated ridge or ridge or row comprising pins or hooks, which ridge lies transverse to the belt direction and to which a belt end can be fastened onto with heat melting or gluing.
49. Any one of the above or below connectors wherein the ridge member has pins or hooks that grab onto the belt fabric without need of heating.
50. Any one of the above or below connectors wherein the frame has an adjustment slot for a user to adjust the belt, which is configured with either of the two described fastening means, the adjustment slot having a row of teeth, pins or hooks transverse to the belt direction, through which adjustment slot a loop of said belt is inserted, which hooks onto the teeth/pins when pulled on, such that the length of the belt is readily adjusted but also secured in the desired adjusted length.
51. Any one of the above or below connectors wherein the connector includes a cover substantially or essentially fully enclosing the frame, which cover either includes a pre-made hole overlapping the receiving hole of the frame, or is readily perforated by pressing the connector onto a male fastener which fits the receiving hole of the frame.
52. Any one of the above or below connectors wherein the suitable cover is arranged by a suitably sized paper, plastic or fabric sticker (foldable sheet with glue on one side) which sticker is folded over the frame after the belt end has been fastened and the wire end electrically connected to the receiving hole, or the cover is from but not limited to a paper envelope, a plastic envelope and a textile envelope, which envelope is suitably fastened by gluing, sewing or the like.
53. Any one of the above or below connectors wherein the biometric device has a further male projecting member which fits within the slot of the frame or within a separate mating hole, the cover is suitably arranged with corresponding openings for such hole or slot for receiving such mating male member, and the cover has a suitable hole allowing the protrusion of a male protruding member being a part of the frame which fits in a mating receiving hole or slot on the mating biometric device.
54. Any one of the above or below connectors wherein the connector comprises a shield member which is arranged on the rear side of the frame to electrically shield the wire from the rear side exterior of the belt connector.
55. Any one of the above or below connectors wherein the shield member is a sheet member extending from the frame, which sheet member is configured to be folded over onto the rear side of the frame to cover said hole and engaged wire.
56. Any one of the above or below connectors wherein the shield member is molded in one piece with the frame with enough strength but suitably flexible to allow folding at least once without braking allows the use of a cover enclosing the frame, which cover need not be electrically insulating, as the shield insulates the only part of the connector which could conduct electrical current of the connector, except through the hole.
57. Any one of the above or below connectors wherein an end portion 109 of an electrode wire from belt 2 is arranged to extend across or bypass a portion of the receiving hole 106 defined in frame 103 by first member 108 and/or second member 113.
58. Any one of the above or below connectors wherein the end portion of electrode wire 109 is arranged to extend across the receiving hole 106 on the rear side 105 of the frame 103 (the rear side 105 being the side of the frame 103 that is opposite of the frame from the front side 104 of the frame 103 that is positioned in contact with the biometric device).
59. Any one of the above or below connectors wherein the belt connector 101 is configured such that when the male snap portion penetrates the hole 106, radial flexibility of first and second members 108 and 113 fasten the frame to male snap portion, and upon fastening the frame to the male snap portion, a contact portion 119 of the wire is forced into contact with a distal portion of the male snap portion.
60. Any one of the above or below connectors wherein the contact portion 119 of the wire is forced against the top surface 170 or a surface of the top bevel 169 of the male snap portion 151 or both.
61. Any one of the above or below connectors wherein a reliable electrical connection between the contact portion 119 and the male snap portion 151 is secured.
62. Any one of the above or below connectors wherein the electrode wire 109 is formed into a looped portion 121 to provide additional surface area as the contact portion 119 of the wire.
63. Any one of the above or below connectors wherein an inside surface of the shield 110 or an additional lid provide structure to force the contact portion 119 of the electrode wire into secure contact with the male snap portion 151.
64. Any one of the above or below connectors wherein the contact portion 119 of the electrode wire is held in place bypassing or extending across the receiving hole 106 through various means.
65. Any one of the above or below connectors wherein a contact member 101 is formed adjacent or near hole 106 and is configured to hold the contact portion 119 of the electrode wire in position adjacent to the hole.
66. Any one of the above or below connectors wherein the contact member 101 has a lateral surface 102 configured to exert a lateral force onto an area of contact portion 119 of the electrode wire, thus, the contact portion 119 of the electrode wire is maintained in position.
67. Any one of the above or below connectors wherein multiple contact members 101 is formed or defined by frame 103 to hold contact portion 119 of electrode wire 109 to extend across a rear side 104 of said frame 103.
68. Any one of the above or below connectors wherein a contact portion 119 is held in place by an adhesive applied to a portion of first member 108 or second member 113.
69. Any one of the above or below connectors wherein a groove or recess portion is formed in a portion of the frame adjacent to hole 106.
70. Any one of the above or below connectors wherein a groove or recess portion is formed in a portion of the frame adjacent to hole 106 on the rear side 105 of first member 108 or 113, or both.
71. Any one of the above or below connectors wherein the groove or recess portion is configured to at least partially envelop a portion of electrode wire 109 to maintain a contact portion 119 of the electrode wire to extend across or bypass the receiving hole 106.
72. Any one of the above or below connectors wherein a contact member, adhesive, or groove me similar be formed or applied ton an inside surface of shield 110 or an additional lid structure, to maintain the contact portion 119 in place against the top surface 170 of the male snap protrusion 151.
73. Any one of the above or below connectors wherein a contact portion 219 is held on a front side 204 of the frame 203 (the side of the frame facing toward the biometric device).
74. Any one of the above or below connectors wherein the contact portion 219 of electrode wire end 209 extends substantially along the front side 204 of second member 213.
75. Any one of the above or below connectors wherein, when the male snap portion is inserted and snapped into hole 206, the contact portion 219 of the electrode wire end 209 is forced into contact with the surface of the male snap foundation 161, or a portion of the lateral surface of the protrusion shaft 167, or both.
76. Any one of the above or below connectors wherein a reliable and repeatable electrical connection between the contact portion 219 of the electrode wire end 209 and the conductive male snap portion is secured upon inserting and fastening the male snap portion 151 into receiving hole 206.
77. Any one of the above or below connectors wherein contact members 201 extend from frame 203 in a direction substantially perpendicular to the electrode wire end 209.
78. Any one of the above or below connectors wherein Contact members 201 each have lateral surfaces 202 configured to hold the contact portion 219 in place near the receiving hole 206.
79. Any one of the above or below connectors wherein electrode wire end 209 is formed to have a looped portion 221 to provide additional wire and area to contact portion 219 to contact the surface of male snap foundation 161.
80. Any one of the above or below connectors wherein, in addition to, or alternatively to, the contact members 201, the contact portion 219 of the electrode wire 209 is held or secured in place by an adhesive 225 applied to second member 213.
81. Any one of the above or below connectors wherein, alternatively, or in addition to the contact members 201 or an applied adhesive, a groove or recessed portion 230 is formed in a surface of first or second member, the groove or recessed portion 230 being arranged to at least partially hole or secure the contact portion 219 of electrode wire 209 in place.
82. Any one of the above or below connectors wherein first member 208 and second member 213 provide a resilient force not only to the male protrusion 151 when it penetrates through receiving hole 206, but first member 208 and second member 213 also provide a contact force to push the contact portion 219 into a secure and reliable connection with a surface of the male snap foundation 161, or a portion of the lateral surface of the protrusion shaft 167, or both.
83. Any one of the above or below connectors wherein a contact portion 319 of electrode wire 309 is held in place on a rear side 305 of frame 303, such that when the male protrusion 151 penetrates through receiving hole 306, the contact portion 319 is forced to the side of the hole 306 such that at least a portion of lateral surface 167 of protrusion shaft 166, a lateral surface 177 of bulged portion 175, a surface of top bevel 169, and/or a surface of underside bevel 168 is forced into contact with contact portion 319.
84. Any one of the above or below connectors wherein contact portion 319 is held in position by structural support from an inside surface of shield 310.
85. Any one of the above or below connectors wherein contact members 301 having lateral surfaces 302 are provided to hold the contact portion 319 in place and exert a lateral force upon contact portion 319 when the male snap portion 151 penetrates hole 306.
86. Any one of the above or below connectors wherein contact members 301 are formed of various shapes so long as they provide the lateral support and force.
87. Any one of the above or below connectors wherein there is a single contact members or two or more contact members 301 are provided.
88. Any one of the above or below connectors wherein, in addition to, or alternatively to, the contact member(s) 301, the contact portion 319 of the electrode wire 309 is held or secured in place by an adhesive applied to first or second member.
89. Any one of the above or below connectors wherein, alternatively, or in addition to the contact members 301 or an applied adhesive, a groove or recessed portion are formed in a surface of first or second member 308, 313, the groove or recessed portion being arranged to at least partially hold or secure the contact portion adjacent to the rear side of the receiving hole 306.
90. Any one of the above or below connectors wherein, alternatively, a contact member, adhesive, or groove, or other means, are provided on an inside surface of shield 310 or another lid structure to maintain the contact portion 319 in lateral contact with the male snap portion 151 when inserted into hole 306.
91. Any one of the above or below connectors wherein a conductive connector or bridging conductor is used to contact a surface of the male snap portion.
92. Any one of the above or below connectors wherein the conductive connector includes an electrical crimp shoe, including a wire connector 405 and a contact portion 406, the wire connector 405 being configured to be connected (such as by crimping) to the electrode wire of the belt.
93. Any one of the above or below connectors wherein the contact portion is arranged with the belt connector frame in such a way to contact a surface of a male snap portion.
94. Any one of the above or below connectors wherein the belt connector provides a reliable and easily repeatable connection between a biometric device and a wire belt electrode.
95. Any one of the above or below connectors wherein the frame 503 is provided having a rear side 505.
96. Any one of the above or below connectors wherein the frame includes a first member 508 and a second member 513 that define a receiving hole 506.
97. Any one of the above or below connectors wherein the first member 508 and second member 513 further define a first slot 511, and additionally define a second slot 515.
98. Any one of the above or below connectors wherein the frame 503 further includes a belt slot with teeth 517 configured to attach the frame 503 to a belt 2.
99. Any one of the above or below connectors wherein a conductive connector 525 is be provided and coupled at least partially to the surface of the frame 503 at the rear side 505 of the frame (opposite from the biometric device).
100. Any one of the above or below connectors wherein conductive connector 525 includes a contact plate that covers, at least partially or entirely, the receiving hole 506.
101. Any one of the above or below connectors wherein conductive connector 525 further includes a wire connector 527, which are a wire crimp configured to be crimped onto an electrode wire end of belt 2.
102. Any one of the above or below connectors wherein the contact plate 526 has a contact surface 528 configured to at least partially contact a top surface 170 or a portion of top bevel 169, or both, of the male snap portion 151 when the male snap portion is inserted and fastened within receiving hole 506.
103. Any one of the above or below connectors wherein, when the male snap protrusion is inserted into receiving hole 506, first member 508 and second member 513 provide a radial flexibility to the receiving hole 506, which causes the male snap portion 151 to be securely held within receiving hole 506.
104. Any one of the above or below connectors wherein frame 503 includes a shield or lid portion attached to the frame body by one or more flexible hinges.
105. Any one of the above or below connectors wherein the shield or lid portion provides additional support to a surface of contact plate 526 opposite from the contact surface 528, to provide a more secure electrical connection between the contact plate 526 and the tope surface 170 or a portion of top bevel 169 of male snap protrusion 151.
106. Any one of the above or below connectors wherein conductive connector 525 is coupled to frame 503 by an adhesive.
107. Any one of the above or below connectors wherein conductive connector 525 is coupled to frame 503 through a crimping mechanism.
108. Any one of the above or below connectors wherein contact plate 526 is bent to have a concavity facing away from the frame 503, such as to provide a spring force in an opposite direction to the insertion direction of the male snap portion 151 into the hole 506.
109. Any one of the above or below connectors wherein a spring force provides a secure and solid contact of the contact plate 526 against the top portions of the male snap portion 151 during movement.
110. Any one of the above or below connectors wherein the conductive connector 601 includes a contact portion 606 and a wire connector 627.
111. Any one of the above or below connectors wherein the contact portion 606 includes a rectangular cut-out 662 configured to couple or contact a top portion (top surface 170 or a portion of top bevel 169) of the male snap portion.
112. Any one of the above or below connectors wherein the rectangular cut-out 662 is configured to mate with a male snap portion having a rectangular or square cross-sectional area or rectangular or square top surface.
113. Any one of the above or below connectors wherein a circular cut-out 661 is provide configured to mate with a male snap portion having a circular cross-sectional area, or a circular top surface.
114. Any one of the above or below connectors wherein the conductive connector is forked-shaped, with a base portion and two arms that extend parallel to each other from the base portion.
115. Any one of the above or below connectors wherein the fork-shaped conductive connector is configured to be fit on a rear side 605 of the frame 603, and is arranged between the frame 603 and a lid or shield.
116. Any one of the above or below connectors wherein with the fork-shaped conductive connector, an internal portion of the arms is forced into contact with a top portion of the male snap protrusion 151.
117. Any one of the above or below connectors wherein a frame 703 having a front side 704 and a rear side 705, are configured to be coupled to a fork-shaped conductive connector 725 on the front side 704 of frame 703.
118. Any one of the above or below connectors wherein the fork-shaped conductive connector 725 includes a base portion 729, a first arm 731, and a second arm 732.
119. Any one of the above or below connectors wherein the first arm 731 is substantially parallel to the second arm 732.
120. Any one of the above or below connectors wherein the conductive connector 725 further includes a wire connector 727 (such as wire crimp), configured to connect the conductive connector, both physically and electrically, to the electrode wire end 709 of the electrode belt.
121. Any one of the above or below connectors wherein each, or at least one of, first arm 731 and/or second arm 732 has a contact surface 733, 734, configured to contact a surface of male snap foundation 161, a lateral surface 167 of protrusion shaft 165, or both.
122. Any one of the above or below connectors wherein a first arm 731 is provide between first member 708 and a portion of male snap foundation 161, and/or second arm 732 is provided between second member 713 another portion of male snap foundation 161, when the male snap portion 151 is inserted through hole 706.
123. Any one of the above or below connectors wherein the underside bevel 168, in conjunction with the radial flexibility of the first member 708 and 713 provides flexibility transvers to their longitudinal axis, thus forcing the first arm 731 and the second arm 732 into secure contact with the male snap foundation 161.
124. Any one of the above or below connectors wherein the conductive connector is a rectangular ring-shaped conductive connector 751, with a wire connector 757.
125. Any one of the above or below connectors wherein the ring-shaped conductive connector 751 are configured to be placed on a front side 704 of the frame 703, similar to the fork-shaped conductive connector, to be placed into secure physical and electrical contact with the male snap foundation 161.
126. Any one of the above or below connectors wherein the first arm 781 and second arm 782 of fork-shaped conductive connector 780 extends from base portion 779 and is bent to form a concavity facing toward the frame 703, such that a spring force is provided between first arm 781 and second arm 782.
127. Any one of the above or below connectors wherein a secure contact is provided between contact surfaces 783 and 784 (of first arm 781 and second arm 782, respectively) and the male snap foundation 161.
128. Any one of the above or below connectors wherein conductive connector 780 includes a wire connector (such as a crimp) to physically an electrically connect the conductive connector to a wire electrode of the electrode belt.
129. Any one of the above or below connectors wherein a conductive connector 825 is provided with a contact portion 826 configured to at least be partially inserted into receiving hole 806.
130. Any one of the above or below connectors wherein frame 803 includes first member 808 and second member 813 that define lateral sides of receiving hole 806.
131. Any one of the above or below connectors wherein first member 808 and second member 813 define first slot 811 and second slot 815.
132. Any one of the above or below connectors wherein frame 803 has a front side 804 (configured to face toward the biometric device) and a rear side 805. Frame 803 is configured to be coupled with conductive connector 825.
133. Any one of the above or below connectors wherein a conductive connector includes body portion 829 extending from the wire connector 827 to the contact portion 826.
134. Any one of the above or below connectors wherein contact portion 826 is arranged (such as by bending) to extend in a plane perpendicular or nearly perpendicular to the direction in which the body portion 829 extends.
135. Any one of the above or below connectors wherein contact portion 826, which is be shaped as a flag, thus is inserted and attached into receiving hole 806 such that when male snap portion 151 is inserted into hole 806, a contact surface of contact portion 826 be brought into physical and electrical contact with a surface of male snap portion 151.
136. Any one of the above or below connectors wherein radial flexibility of at least one of the first member 808 and second member 813 provides a lateral force substantially perpendicular to the longitudinal axis of the first member 808 or second member 813 as to force the contact portion securely against a lateral surface of male snap portion 151.
137. Any one of the above or below connectors wherein conductive connector 825 is coupled to frame 803 by a crimping of the conductive connector to the frame, an adhesive, tension fitting, heat fitting, shrink fitting, a fastener, or other means of connection.
138. Any one of the above or below connectors wherein a U-shaped conductive connector 925 is provided.
139. Any one of the above or below connectors wherein first member 908 and second member 913 are provided adjacently to and define at least in part receiving hole 906.
140. Any one of the above or below connectors wherein receiving hole 906 is rectangular in shape, so as to receive and mate with the cross-sectional top plan shape of the U-shaped conductive connector 925.
141. Any one of the above or below connectors wherein the U-shaped conductive connector is inserted into frame 903 and wire connector 927 is connected to electrode wire 909, by crimping, soldering, or fastening.
142. Any one of the above or below connectors wherein U-shaped conductive connector 925 is arranged with the opening of the U-shape, or the concavity of the U-shape to be pointed toward the front side 904 of the frame 903 such that the male snap portion 151 are inserted into the opening formed by the U-shaped conductive connector 925.
143. Any one of the above or below connectors wherein in a cross sectional view, U-shaped conductive connector has first side portion 931 and an opposing second side portion 932, each extending from base portion.
144. Any one of the above or below connectors wherein the first side portion 931 has a first contact surface 933 on the inside surface and second side portion 932 has a first contact surface 934 on the inside surface of second side portion 932.
145. Any one of the above or below connectors wherein, through the lateral force provided by the first member 908 and/or the lateral force provided by the second member 913, the first contact surface 931 and second contact surface 934 are forced into secure contact with a lateral surface of male snap portion 151.
146. Any one of the above or below connectors wherein, a restorative spring force of the first side portion 931 and second side portion 932 provide a lateral force to hold first contact surface 931 and second contact surface 934 in contact with a lateral surface of male snap portion 151.
147. Any one of the above or below connectors wherein on a side of the conductive connector 925 opposite from the direction in which the first and second side portions extend, a shield (not shown) or lid attached to the rear side of the frame 903 gives additional support in the direction toward the biometric device.
148. Any one of the above or below connectors wherein respective lip portions 935, 936, of first and second side portions 931, 932, extend onto a surface of front side 905 of the frame 903, so as to maintain the conductive connector 925, particularly when male snap portion 151 is inserted within the U-shaped conductive connector and hole 906.
149. Any one of the above or below connectors wherein a frame 1003 has a front side 1004 and a rear side 1005.
150. Any one of the above or below connectors wherein the frame includes a first member 1008 and a second member 1013, which define at least partially, a receiving hole 1006 with radial flexibility.
151. Any one of the above or below connectors wherein first member 1008 and 1013 further define first slot 1011 and second slot 1015, which contribute to the radial flexibility provided at receiving hole 1006.
152. Any one of the above or below connectors wherein a top spring conductive connector 1025 is provided having a wire connector 1027, a first contact member 1031, and a second contact member 1032.
153. Any one of the above or below connectors wherein the top spring conductive connector is arranged on a rear side 1005 of frame 1003, such that when a male snap portion 151 is inserted within receiving hole 1006, lateral surfaces of male snap portion are brought and held in contact with inner contact surfaces of conductive connector 1025, including first contact surface 1033 of first contact member 1031 and first contact surface of second contact member 1032.
154. Any one of the above or below connectors wherein additionally, electrical contact is made between an inner surface of the base portion 1035 of the conductive connector and a top surface 170 of the male snap portion 151.
155. Any one of the above or below connectors wherein the male snap portion 151 is securely held fastened to the belt connector due to the radial flexibility provided by first and second elongated members 1008 and 1013, and is also held in place by the spring force provided by the first contact member 1031 and the second contact member 1032.
156. Any one of the above or below connectors wherein conductive connector 1025 are coupled to frame 1003 by a crimping of the conductive connector 1025 to the frame, an adhesive, tension fitting, heat fitting, shrink fitting, a fastener, or other means of connection.
157. Any one of the above or below connectors wherein wire connector 1027 provides an electrical and physical connection between the conductive connector 1025 and the electrode wire end 1009.
158. Any one of the above or below connectors wherein radial flexibility of the receiving hole is provided by the first member and the second member, which define at least partially the receiving hole and slots, such as a first slot and second slot, the second slot being arranged on a different side of the receiving hole from the first slot.
159. Any one of the above or below connectors wherein the receiving hole has radial flexibility due only to the material surrounding the receiving hole.
160. Any one of the above or below connectors wherein a frame is formed entirely or partially from an elastic material, such a rubber or other elastic polymer.
161. Any one of the above or below connectors wherein a frame includes a portion, such as elongated members, formed of a molded and more firm plastic, while inner portion adjacent to the receiving hole, are formed of a softer, more flexible and resilient material.
162. Any one of the above or below connectors wherein a frame is combined with the above-described configurations to provide a belt connector with a conductive connector or wire configured to securely contact a surface (either top, lateral, or foundation) of a corresponding male snap portion.

1. An electrode belt connector for electrically connecting a conductor of an electrode belt to a first snap portion of a snap connector electrode configured to be connected to a biometric device, the electrode belt connector comprising:
a frame defining a corresponding second snap portion configured to mate with the first snap portion of the snap connector electrode; and
a connecting portion configured to electrically connect the conductor of the electrode belt to the first snap portion of the snap connector electrode.

2. The belt connector according to claim 1, wherein the first snap portion is a male snap portion.

3. The belt connector according to claim 1, wherein the second snap portion is a receiving hole.

4. The belt connector according to claim 1, wherein the electrode belt connector is configured to connect ends of a respiratory inductive plethysmography belt that includes said wire woven within the respiratory inductive plethysmography belt.

5. The belt connector according to claim 1, wherein the frame includes a first member and a second member that define a receiving hole between the first member and the second member,
wherein the first member and the second member are configured to provide a radial resilient force to a male snap protrusion of the first snap portion when the male snap protrusion is inserted into said receiving hole.

6. The belt connector according to claim 5, wherein the radial resilient force forces the connecting portion to electrically connect an electrically conductive portion to electrically connect the male snap protrusion inserted into said receiving hole.

7. The belt connector according to claim 5, wherein the connecting portion includes a conductive connector that passes through or above said receiving hole, or the connecting portion is an end portion of the wire of the electrode belt that passes through or above said receiving hole.

8. The belt connector according to claim 5, wherein the connecting portion includes a conductive foil connector configured to electrically connect the wire of the electrode belt to the first snap portion of the snap connector.

9. The belt connector according to claim 8, wherein connecting portion is provided such as to be between the frame and the biometric device.

10. The belt connector according to claim 1, wherein the connecting portion includes a conductive connector configured to connect the wire of the electrode belt to the first snap portion of the snap connector.

11. The belt connector according to claim 1, wherein the connecting portion one or more contact members configured to connect the wire of the electrode belt to the first snap portion of the snap connector.

12. The belt connector according to claim 1, wherein the connecting portion includes an electrically conductive connector configured to connect the wire of the electrode belt to the first snap portion of the snap connector.

13. The belt connector according to claim 1, wherein the connecting portion includes an electrically conductive portion that pass through or above said receiving hole.

14. The belt connector according to claim 1, wherein the frame includes a front side configured to face toward the biometric device and a rear side configured to face away from the biometric device, and
wherein the connecting portion is arranged on the rear side the frame.

15. The belt connector according to claim 1, wherein the connecting portion includes an electrically connecting portion provided such as to be between the frame and the biometric device.
